(19) United States
(12) Patent Application Publication
(10) Pub. No.: US 2025/0082277 A1
(43) Pub. Date: Mar. 13, 2025

(54) CARDIAC SIGNAL QUALITY CHECK AND FEATURE EXTRACTION PIPELINE USING MORPHOLOGICAL FEATURES FOR STRESS LEVEL ESTIMATION

(71) Applicant: SAMSUNG ELECTRONICS CO., LTD., Suwon-si (KR)

(72) Inventors: Viswam NATHAN, Clovis, CA (US); Larry ZHANG, San Jose, CA (US); Md Mahbubur RAHMAN, San Jose, CA (US); Jilong KUANG, San Jose, CA (US); Jungmok BAE, Menlo Park, CA (US); Jun GAO, Menlo Park, CA (US)

(73) Assignee: SAMSUNG ELECTRONICS CO., LTD., Suwon-si (KR)

(21) Appl. No.: 18/802,677

(22) Filed: Aug. 13, 2024

Related U.S. Application Data
(60) Provisional application No. 63/538,269, filed on Sep. 13, 2023.

(51) Int. Cl. A61B 5/00; A61B 5/024; A61B 5/0295; A61B 5/16

(52) U.S. Cl. CPC A61B 5/7267; A61B 5/02405; A61B 5/0295; A61B 5/165; A61B 5/681; A61B 5/6817; A61B 5/6898; A61B 5/7203; A61B 5/7221; A61B 5/7225; A61B 5/7239; A61B 5/7278; A61B 5/7405; A61B 5/743; A61B 5/7455; A61B 2560/0204; A61B 2560/0431; A61B 2560/045; A61B 2560/0462

(57) ABSTRACT

A computer-implemented method includes: receiving, from a sensor of an electronic device, a photoplethysmography (PPG) signal about a user; dividing the received PPG signal into a first set of waveforms; checking qualities of the first set of waveforms and generating a second set of waveforms based on the checked qualities of the first set of waveforms; extracting a plurality of morphological features of each waveform of the second set of waveforms and calculating a plurality of time/amplitude factors based on the extracted plurality of morphological features; estimating, using an artificial intelligence model, a stress level of the user, based on the calculated plurality of time/amplitude factors; and providing the estimated stress level to the user.

### CROSS-REFERENCE TO RELATED APPLICATIONS

This application is based on and claims priority under 35 U.S.C. § 119 to U.S. Provisional Patent Application No. 63/538,269, filed on Sep. 13, 2023, in the U.S. Patent and Trademark Office, the disclosure of which is incorporated by reference herein in its entirety.

### BACKGROUND

### 1. Field

The disclosure relates to a method for detecting stress using morphological photoplethysmography features of wearable devices, for example, multimodal earbuds having sensors, and relates to the same wearable devices.

### 2. Description of Related Art

Continuous monitoring of stress has become a focal interest in health sensing, due to the mental and physical effects of long-term stress. Recent work demonstrated the feasibility of photoplethysmography (PPG) based heart rate variability (HRV) features from wearable devices (in particular, multimodal earbuds having sensors) to detect stress. However, morphological PPG features from the wearable devices have not been evaluated for stress detection.

Stress is characterized by physiological changes in response to mental and environmental stimuli. Physiological responses to stress range from benign to malignant, as some forms of stress are beneficial for well-being, while other forms can be detrimental. Chronic malignant stress can result in negative health effects including cardiovascular disease, gastrointestinal distress, and general hypertension. Continuous monitoring of stress can enable better stress management, leading to improved well-being and health. Recently, there has been a growing interest in using wearable devices (e.g., multimodal earbuds having sensors) to better characterize physiological stress.

So far, the majority of stress monitoring research focuses on HRV analysis in PPG and electrocardiography (ECG) signals. HRV provides unique insight into autonomic nervous system (ANS) function, where physiological changes are mediated through the sympathetic nervous system (SNS) and parasympathetic nervous system (PNS). The primary function of the SNS is to oxygenate blood, enabling mobilization of energy and resources in the body. The primary function of the PNS is to modulate the activation of the SNS allowing the body to restore itself following sympathetic activation.

In complement to HRV, PPG morphology may also be useful for stress monitoring. Morphological features may be more sensitive to fine-grained changes in physiological responses as the morphological features measure changes at the individual PPG pulse wave level. Previous studies described the effect of mental stress on PPG morphology. The length of pulse waves and time to systolic peak were shown to decrease during stress periods in comparison to baseline. Additionally, the pulse wave width was shown to decrease in response to stressors. PPG morphology can also be used to estimate other physiological dimensions related to stress, such as blood pressure. Based on the preliminary evidence, PPG morphology may complement HRV features in stress detection.

There have been numerous studies evaluating the use of PPG-based HRV features for stress detection. FIG. 1 shows an example of the PPG-based HRV features of the related art, in which a PPG pulse wave has nine interbeat intervals (IBIs). FIG. 1 also shows HRV metrics known and used in the related art: root mean square of successive RR interval differences (RMSSD, unit: ms), standard deviation of the IBI of normal sinus beats (SDNN, unit: ms), hear rate (HR, unit: bpm), respiratory sinus arrhythmia (RSA). Optionally, a bandpass filter with a central tendency dictated by the heartrate of the segment estimated via the Fast Fourier Transform of the signal (FFT), and a tolerance range of +0.2 Hz to −0.2 Hz is used before the HRV features are extracted from the PPG pulse wave.

The PPG-based HRV features of the related art are prone to errors or loss of data since a single missing or wrongly estimated IBI in the IBI series can have a large effect on the downstream HRV feature estimation. Even if erroneous IBIs are identified and removed by methods such as outlier detection, the derived HRV still has error since the IBI chain is broken. Thus, the PPG-based HRV features may need to be replaced with other or be used with other features such as PPG morphology based features.

However, studies on the use of the PPG morphology based features for stress detection have been sparse. A number of PPG morphology based stress detection studies focused on stress monitoring in driving scenarios, while the most recent study focused on differentiating stress from amusement and neutral states in a laboratory setting.

Sensors on wearable devices (or wearable sensors) may implement stress monitoring solutions. The majority of stress monitoring solutions implemented in the wearable sensors are based on HRV analysis in PPG and ECG signals. There are a number of problems with the majority of stress monitoring solutions.

First, the estimation of HRV accurately depends on precise detection of inter-beat intervals (IBIs), which can be challenging especially in the PPG signal. Even a small error in the peak detection of PPG can lead to a large error in HRV, which in turn, can have downstream effects on the accuracy of stress classification.

Second, wearable sensors are resource constrained, and also need to be mindful of power consumption, which means lower sampling rates which in turn makes it even more difficult to extract the peaks accurately for IBI-based HRV approaches,

Third, HRV based approaches do not take advantage of morphological changes in the PPG waveform due to stress, and only consider the time interval between waves, thus ignoring a rich source of information.

Fourth, the few approaches that use the morphological features of PPG are built on very high sampling rate PPG sampled at a site such as a fingertip in order to obtain a high-fidelity waveform with all the features of the PPG such as the dicrotic notch. However, PPG from commercial wearable devices located in the ear canal or wrist do not elicit detailed waveforms due to the use of reflective PPG with green LED that captures only the capillary action. Moreover, commercial devices need to restrict sampling rate due to battery life constraints on wearable devices.

Fifth, the signal quality is generally lower for wearable sensors due to various noise sources in everyday life (for example, motion artifacts or a loosely worn sensor), and existing methods either suffer from noise or can only measure stress during ideal monitoring conditions.

### SUMMARY

One or more embodiments of the disclosure are directed to an analysis of physiological data from periods of stress and non-stress. Further, one or more embodiments of the disclosure are directed to a training of random forest models on morphological, PPG HRV, and ECG HRV features. The morphological features may outperform PPG HRV features. The combination of PPG morphological features and HRV features performed similarly to ECG HRV features, which suggests that PPG morphological and HRV features from wearable devices (e.g., multimodal earbuds having sensors) are able to detect stress with similar fidelity to ECG despite the smaller form factor. Thus, the wearable devices (e.g., multimodal earbuds having sensors) of the disclosure may perform continuous monitoring of physiology.

Particular applications of embodiments of the disclosure may include: 1) an improved accuracy or precision for detecting stress, compared with the accuracy or the precision of the existing methods (e.g., HRV-based feature extraction shown in FIG. 1); 2) an improved ability of the electronic devices for measuring stress even during periods when the PPG signal is too noisy for the existing methods; and 3) an improvement of stress detection capability (in terms of accuracy or precision) of the electronic devices (e.g., the wearable devices) having relatively low sampling rates (e.g., 25 Hz) due to their constrained resources.

According to one aspect of the disclosure, a computer-implemented method includes: receiving, from a sensor of an electronic device, a photoplethysmography (PPG) signal about a user; dividing the received PPG signal into a first set of waveforms; checking qualities of the first set of waveforms and generating a second set of waveforms based on the checked qualities of the first set of waveforms; extracting a plurality of morphological features of each waveform of the second set of waveforms and calculating a plurality of time/amplitude factors based on the extracted plurality of morphological features; estimating, using an artificial intelligence model, a stress level of the user, based on the calculated plurality of time/amplitude factors; and providing the estimated stress level to the user.

According to an aspect of the disclosure, an electronic device includes: a sensor; at least one memory; at least one processor operatively connected with the sensor and the at least one memory, the at least on processor configured to: receiving, from the sensor, a photoplethysmography (PPG) signal about a user; divide the received PPG signal into a first set of waveforms; check qualities of the first set of waveforms and generate a second set of waveforms based on the checked qualities of the first set of waveforms; extract a plurality of morphological features of each waveform of the second set of waveforms and calculate a plurality of time/amplitude factors based on the extracted plurality of morphological features; estimate, using an artificial intelligence model, a stress level of the user, based on the calculated plurality of time/amplitude factors; and provide the estimated stress level to the user.

According to an aspect of the disclosure, a computer-implemented method includes: receiving, from a sensor of an electronic device, a photoplethysmography (PPG) signal about a user; determining whether a noise of the PPG signal is equal to or higher than a threshold; based on a determination that the noise of the PPG signal is equal to or higher than the threshold, dividing the received PPG signal into a first set of waveforms; checking qualities of the first set of waveforms and generating a second set of waveforms based on the checked qualities of the first set of waveforms; extracting a plurality of morphological features of each waveform of the second set of waveforms and calculating a plurality of time/amplitude factors based on the extracted plurality of morphological features; estimating, using an artificial intelligence model, a stress level of the user, based on the calculated plurality of time/amplitude factors; and providing the estimated stress level to the user.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates an example of a photoplethysmography (PPG) pulse wave having nine interbeat interval (IBIs) in the related art of heart rate variability (HRV) feature extraction pipeline;

FIG. 2 illustrates example components of an electronic device in accordance with embodiments of the disclosure;

FIG. 3A illustrates a first example (a pair of earbuds) of the electronic device in accordance with some embodiments of the disclosure;

FIG. 3B illustrates example components of the first example of the electronic device in accordance with some embodiments of the disclosure;

FIG. 4A illustrates a second example (a watch) of the electronic device in accordance with some embodiments of the disclosure;

FIG. 4B illustrates example components of the second example of the electronic device in accordance with some embodiments of the disclosure;

FIG. 5A illustrates a third example (a ring) of the electronic device in accordance with some embodiments of the disclosure;

FIG. 5B illustrates example components of the third example of the electronic device in accordance with some embodiments of the disclosure;

FIG. 6 illustrates example structures of a sensor for detecting biometric information according to one or more example embodiments;

FIG. 7 illustrates an example system having a fourth example (a computer) of the electronic device with remotely located electronic devices and their sensors in accordance with some embodiments of the disclosure;

FIG. 8A illustrates a series of operations (a “pipeline”) performed by electronic devices in accordance with some embodiments of the disclosure;

FIG. 8B illustrates a PPG waveform and a first derivative of the PPG waveform in accordance with some embodiments of the disclosure;

FIG. 9 illustrates good quality waveforms and poor quality waveforms;

FIG. 10 illustrates an example waveform of the PPG signal in accordance with some embodiments of the disclosure;

FIG. 11 illustrates a configuration of combining the HRV feature extraction pipeline with the PPG morphological feature extraction pipeline in accordance with some embodiments of the disclosure;

FIG. 12 illustrates a first particular application (a mobile phone) in accordance with some embodiments of the disclosure;

FIG. 13 illustrates a second particular application (a mobile phone and earbuds) in accordance with some embodiments of the disclosure;

FIG. 14 illustrates a third particular application (a mobile phone and a watch) in accordance with some embodiments of the disclosure;

FIG. 15 illustrates a fourth particular application (a mobile phone and a ring) in accordance with some embodiments of the disclosure;

FIG. 16 illustrates a first set of operations in accordance with some embodiments of the disclosure; and

FIG. 17 illustrates a second set of operations in accordance with some embodiments of the disclosure.

### DETAILED DESCRIPTION

The terms as used in the disclosure are provided to merely describe specific embodiments, not intended to limit the scope of other embodiments. Singular forms include plural referents unless the context clearly dictates otherwise. The terms and words as used herein, including technical or scientific terms, may have the same meanings as generally understood by those skilled in the art. The terms as generally defined in dictionaries may be interpreted as having the same or similar meanings as or to contextual meanings of the relevant art. Unless otherwise defined, the terms should not be interpreted as ideally or excessively formal meanings. Even though a term is defined in the disclosure, the term should not be interpreted as excluding embodiments of the disclosure under circumstances.

The electronic device according to one or more embodiments may be one of various types of electronic devices. In some embodiments of the disclosure, the electronic devices may include a portable communication device (e.g., a smartphone), a computer device, a portable multimedia device, a portable medical device, a camera, or a wearable device. The electronic devices are not limited to those described above, in accordance with some other embodiments of the disclosure.

The disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B”, “at least one of A and B”, “at least one of A or B”, “A, B, or C”, “at least one of A, B, and C”, and “at least one of A, B, or C”, may include any one of, or all possible combinations of the items enumerated together in a corresponding one of the phrases. As used herein, such terms as “1st” and “2nd”, or “first” and “second” may be used to simply distinguish a corresponding component from another, and does not limit the components in other aspects (e.g., importance or order). It is to be understood that if an element (e.g., a first element) is referred to, with or without the term “operatively” or “communicatively”, as “coupled with”, “coupled to”, “connected with”, or “connected to” another element (e.g., a second element), it means that the element may be coupled with the other element directly (e.g., wiredly), wirelessly, or via a third element.

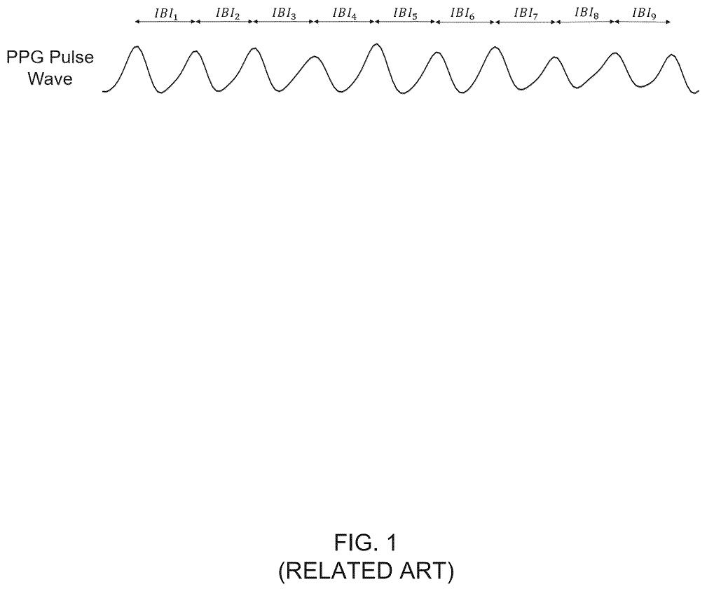

The disclosure is directed to one or more embodiments having a stress classification pipeline (a set of operations) that utilizes the morphological features of the photoplethysmography (PPG) waveform that has equal or better performance than using the heart rate variability (HRV) based features in the related art (for example, as shown in FIG. 1). The PPG based morphological features of the disclosure may help overcome the weaknesses in the interbeat interval (IBI)-based HRV extraction mentioned previously.

The pipeline incorporates a novel waveform quality check block, which ensures the morphological features are extracted only when the underlying PPG signal is of sufficient quality. This approach may balance and satisfy two (2) requirements. First, Periods of noise are common in wearable sensing scenarios, and this approach allows us to reject PPG waveforms distorted by noise. Second, existing HRV-based approaches can lead to large errors if even 1 or 2 IBIs in a 1-minute window of PPG are detected incorrectly, or it can lead to large quantities of data being unusable due to missing/incorrect IBIs. In contrast, the one or more embodiments of the disclosure may harvest and save more PPG windows, since the morphological based approach may still estimate stress even when 1 or more PPG waveforms are rejected due to noise in a larger window. That is, a particular application of the disclosure is a more continuous monitoring operation in free-living scenarios.

Both the morphological feature selection and waveform quality check are tuned for low-sampling rate PPG collected by resource constrained wearable devices, which fundamentally may have a different shape and morphology. This has an advantage over existing morphological feature based approaches which rely on power-consuming high sampling rate data collection with devices that are placed in more cumbersome locations such as the fingertip.

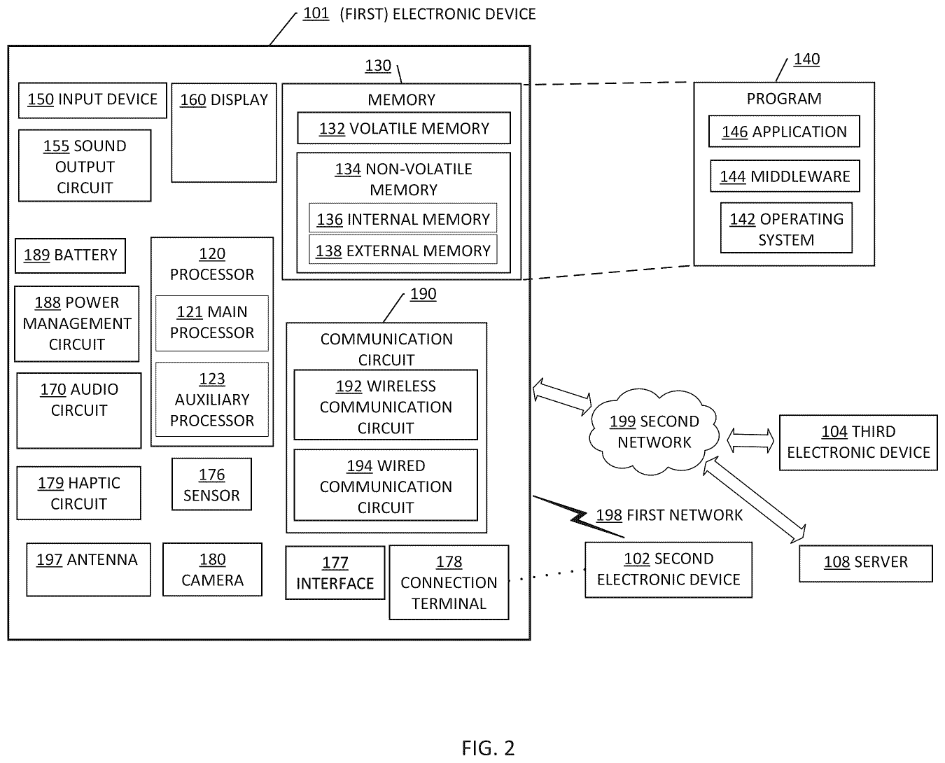

The one or more embodiments of the disclosure may be implemented in or by the following example electronic devices shown in FIG. 2 to FIG. 7.

FIG. 2 illustrates example components of the electronic device in accordance with some embodiments of the disclosure.

In FIG. 2, a (first) electronic device 101 may communicate with a second electronic device 102 via a first network 198 (e.g., a short-range wireless communication network), or a third electronic device 104 or a server 108 via a second network 199 (e.g., a long-range wireless communication network). In one embodiment, the (first) electronic device 101 may communicate with the third electronic device 104 via the server 108. Throughout the disclosure, the first electronic device 101 is referred to as ‘the electronic device 101.’ Hereinafter, components of the electronic device 101 are described. Those components of the electronic device 101 may be also included in the second electronic device 102 or the third electronic device 104.

In one embodiment, the electronic device 101 may include a processor 120, memory 130, an input device 150, a sound output circuit 155, a display 160, an audio circuit 170, a sensor 176, an interface 177, a haptic circuit 179, a camera 180, a power management circuit 188, a battery 189, a communication circuit 190, a subscriber identification module (SIM) 196, or an antenna 197.

In some embodiments, at least one (e.g., the display 160 or the camera 180) of the components may be omitted from the electronic device 101, or one or more other components may be added in the electronic device 101. In some embodiments, some of the components may be implemented as single integrated circuitry. For example, the sensor 176 (e.g., a fingerprint sensor, an iris sensor, or an illuminance sensor) may be implemented as embedded in the display 160 (e.g., a display).

The processor 120 may execute, for example, software (e.g., a program 140) to control at least one other component (e.g., a hardware or software component) of the electronic device 101 coupled with the processor 120, and may perform various data processing or computation. In one embodiment, as at least part of the data processing or computation, the processor 120 may load a command or data received from another component (e.g., the sensor 176 or the communication circuit 190) in volatile memory 132, process the command or the data stored in the volatile memory 132, and store resulting data in non-volatile memory 134. In one embodiment, the processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a specified function. The processor 120 may refer to or correspond to one or more processors. For example, the electronic device 101 may include two or more processors like the processor 120.

The auxiliary processor 123 may be implemented as separate from, or as part of the main processor 121. The auxiliary processor 123 may control at least some of functions or states related to at least one component (e.g., the display 160, the sensor 176, or the communication circuit 190) among the components of the electronic device 101, instead of the main processor 121 while the main processor 121 is in an inactive (e.g., sleep) state, or together with the main processor 121 while the main processor 121 is in an active state (e.g., executing an application). In one embodiment, the auxiliary processor 123 (e.g., an image signal processor or a communication processor) may be implemented as part of another component (e.g., the camera 180 or the communication circuit 190) functionally related to the auxiliary processor 123.

The memory 130 may store various data used by at least one component (e.g., the processor 120 or the sensor 176) of the electronic device 101. The various data may include, for example, software (e.g., the program 140) and input data or output data for a command related thereto. The memory 130 may include the volatile memory 132 or the non-volatile memory 134. The program 140 may be stored in the memory 130 as software, and may include, for example, an operating system (OS) 142, middleware 144, or an application 146.

One or more embodiments of the disclosure may be implemented as software (e.g., the application 146, the middleware 144, the operating system) including one or more instructions that are stored in the memory 130 (a storage medium) that is readable by the electronic device 101. For example, the processor 120 of the electronic device 101 may invoke at least one of the one or more instructions stored in the memory 130, and execute the at least one of the one or more instructions, with or without using one or more other components under the control of the processor 120. This allows the electronic device 101 to be operated to perform at least one function according to the at least one instruction invoked. The one or more instructions may include a code generated by a compiler or a code executable by an interpreter. The memory 130, which may be a machine-readable storage medium, may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the memory 130 (the storage medium) and where the data is temporarily stored in the memory 130.

In some embodiments, functions related to artificial intelligence (AI) are operated by the processor 120 (or the main processor 121 or the auxiliary processor 123) and the memory 130. The processor 120 (or the main processor 121 or the auxiliary processor 123) may include or may correspond to a general-purpose processor, such as a CPU, an application processor, or a digital signal processor (DSP), a graphics-dedicated processor, such as a graphics processing unit (GPU) or a vision processing unit (VPU), or an artificial intelligence-dedicated processor, such as a neural processing unit (NPU). The processor 120 (or the main processor 121 or the auxiliary processor 123) may control input data to be processed according to predefined operation rules or artificial intelligence models, which are stored in the memory 130. Alternatively, the processor 120 (or the main processor 121 or the auxiliary processor 123) may be an artificial intelligence-dedicated processor including a hardware structure specialized for processing of a particular artificial intelligence model.

The predefined operation rules or the artificial intelligence models are made through training. Here, the statement of being made through training means that a basic artificial intelligence model is trained by a learning algorithm by using a large number of training data, thereby making a predefined operation rule or an artificial intelligence model, which is configured to perform a desired characteristic (or purpose). Such training may be performed in a device itself, in which artificial intelligence according to the disclosure is performed, or may be performed via a separate server or a separate system. Examples of the learning algorithm may include, but are not limited to, supervised learning, unsupervised learning, semi-supervised learning, and reinforcement learning.

The artificial intelligence model may include a plurality of neural network layers. Each of the plurality of neural network layers has a plurality of weight values and performs neural network calculations through calculations between a calculation result of a previous layer and the plurality of weight values. The plurality of weight values of the plurality of neural network layers may be optimized by a training result of the artificial intelligence model. For example, the plurality of weight values may be updated to minimize a loss value or a cost value, which is obtained from the artificial intelligence model during the process of training. An artificial neural network may include a deep neural network (DNN), and examples of the artificial neural network may include, but are not limited to, a random forest model, a convolutional neural network (CNN), a DNN, a recurrent neural network (RNN), a restricted Boltzmann machine (RBM), a deep belief network (DBN), a bidirectional recurrent deep neural network (BRDNN), and deep Q-Networks.

The input device 150 may receive a command or data to be used by other components (e.g., the processor 120) of the electronic device 101, from the outside (e.g., a user) of the electronic device 101. The input device 150 may include, for example, a microphone, a mouse, or a keyboard.

The sound output circuit 155 may output sound signals to the outside of the electronic device 101. The sound output circuit 155 may include, for example, a speaker or a receiver. The speaker may be used for general purposes, such as playing multimedia or playing record, and the receiver may be used for incoming calls. In one embodiment, the receiver may be implemented as separate from, or as part of the speaker.

The display 160 may visually provide information to the outside (e.g., a user) of the electronic device 101. The display 160 may include, for example, a display, a hologram device, or a projector and control circuitry to control a corresponding one of the display, hologram device, and projector. In one embodiment, the display 160 may include touch circuitry adapted to detect a touch, or sensor circuitry (e.g., a pressure sensor) adapted to measure the intensity of force incurred by the touch.

The audio circuit 170 may convert a sound into an electrical signal and vice versa. In one embodiment, the audio circuit 170 may obtain the sound via the input device 150, or output the sound via the sound output circuit 155 or a headphone of an external electronic device (e.g., an electronic device 102) directly (e.g., wiredly) or wirelessly coupled with the electronic device 101.

The sensor 176 may detect an operational state (e.g., power or temperature) of the electronic device 101 or an environmental state (e.g., a state of a user) external to the electronic device 101, and then generate an electrical signal or data value corresponding to the detected state. In one embodiment, the sensor 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. In one embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). In one embodiment, the connecting terminal 178 may include, for example, a HDMI connector, a USB connector, a SD card connector, or an audio connector (e.g., a headphone connector),

The haptic circuit 179 may convert an electrical signal into a mechanical stimulus (e.g., a vibration or a movement) or electrical stimulus which may be recognized by a user via his tactile sensation or kinesthetic sensation. In one embodiment, the haptic circuit 179 may include, for example, a motor, a piezoelectric element, or an electric stimulator.

The camera 180 may capture a still image or moving images. In one embodiment, the camera 180 may include one or more lenses, image sensors, image signal processors, or flashes.

The power management circuit 188 may manage power supplied to the electronic device 101. In one embodiment, the power management circuit 188 may be implemented as at least part of, for example, a power management integrated circuit (PMIC).

The battery 189 may supply power to at least one component of the electronic device 101. In one embodiment, the battery 189 may include, for example, a primary cell which is not rechargeable, a secondary cell which is rechargeable, or a fuel cell.

The communication circuit 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication circuit 190 may include one or more communication processors that are operable independently from the processor 120 (e.g., the application processor (AP)) and supports a direct (e.g., wired) communication or a wireless communication. In one embodiment, the communication circuit 190 may include a wireless communication circuit 192 (e.g., a cellular communication circuit, a short-range wireless communication circuit, or a global navigation satellite system (GNSS) communication circuit) or a wired communication circuit 194 (e.g., a local area network (LAN) communication circuit or a power line communication (PLC) module). A corresponding one of these communication circuits may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a cellular network, the Internet, or a computer network (e.g., LAN or wide area network (WAN)). These various types of communication circuits may be implemented as a single component (e.g., a single chip), or may be implemented as multi components (e.g., multi chips) separate from each other. The wireless communication circuit 192 may identify and authenticate the electronic device 101 in a communication network, such as the first network 198 or the second network 199, using subscriber information (e.g., international mobile subscriber identity (IMSI)) stored in the subscriber identification module 196.

The antenna 197 may transmit or receive a signal or power to or from the outside (e.g., the external electronic device) of the electronic device 101. In one embodiment, the antenna 197 may include one or more antennas, and, therefrom, at least one antenna appropriate for a communication scheme used in the communication network, such as the first network 198 or the second network 199, may be selected, for example, by the communication circuit 190 (e.g., the wireless communication circuit 192). The signal or the power may then be transmitted or received between the communication circuit 190 and the external electronic device via the selected at least one antenna.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

In one embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. In one embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, or client-server computing technology may be used, for example.

The electronic device according to various embodiments may be one of various types of electronic devices. The electronic devices may include, for example, a portable communication device (e.g., a smart phone), a computer device, a portable multimedia device, a portable medical device, a camera, a wearable device, or a home appliance. In one embodiment of the disclosure, the electronic devices are not limited to those described above.

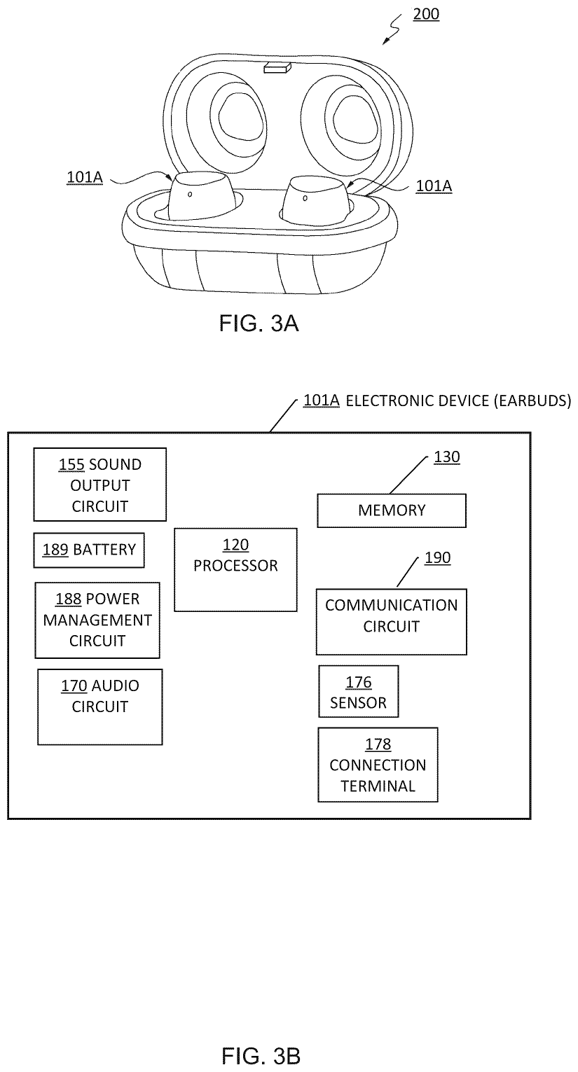

FIG. 3A illustrates an example of the electronic device 101, which is a pair of earbuds 101A with a power supply device 200. FIG. 3A also illustrates an example of the power supply device 200. In some embodiments, the power supply device 200 can have different forms, such as a wall charger. FIG. 3B illustrates that the (pair of) earbuds 101A may include the processor 120, the memory 130, the sound output circuit 155, the audio circuit 170, the sensor 176, the connection terminal 178, the power management circuit 188, the battery 189, and the communication circuit 190. Those components shown in FIG. 3B are examples, that is, some of those components may be omitted or other components (for example, shown in FIG. 2) may be added to the earbuds 101A.

In some embodiments, the earbuds 101A may be paired with a computer, an electronic device, or a mobile device that corresponds to the (first) electronic device 101 shown in FIG. 2. That is, any data processed by the earbuds 101A can be shown on the display 160 of the (first) electronic device 101. In some embodiments, some operations can be shared between the earbuds 101A and the (first) electronic device 101.

In FIG. 3A, when the earbuds 101A is not in use, the earbuds 101A can be accommodated and stored in the power supply device 200, and when the earbuds 101A is in use can be worn and brought into close proximity to a part of the user's body (e.g., ears). In one embodiment, the earbuds 101A may be configured as a pair to be worn on both ears of the user and brought into close proximity.

In one embodiment, the earbuds 101A may output an audio signal (e.g., via the sound output circuit 155) when worn and in close proximity to a part of the user's body. In one embodiment, at least one of a right earbud and a left earbud may output an audio signal using wireless data transmission and reception with an electronic device (such as a mobile phone), for example, configured to play a song to be transmitted to the earbuds 101A.

For example, the wireless data transmission and reception path may be based on or may correspond to a Bluetooth communication scheme, a route for a Bluetooth low energy (BLE) communication scheme, and a route for a ultra-wide band (UWA) communication scheme. The wireless data transmission and reception path may include at least one of a path for a wireless fidelity (Wi-Fi) direct communication technique and a path for a mobile communication technique (e.g., long term evolution (LTE) sidelink, etc.).

In one embodiment, only one of the earbuds 101A may create the communication path with the electronic device. For example, a first earbud may output an audio signal based on the audio data of the electronic device received through the communication path (the wireless data transmission and reception path). When the electronic device is connected to the right earbud, the electronic device or the right earbud provides information about the communication path to the left earbud so that the left earbud can output the audio signal. The left earbud can receive data transmitted to the right earbud based on the information about the communication path and output the audio signal.

For example, the left earbud can receive data transmitted to the right earbud by monitoring information about the communication path. In one embodiment, the right earbud connected to the electronic device may be referred to as a master device, and the left earbud not connected to the electronic device may be referred to as a slave device. In one embodiment, the master device and the slave device among the pair of the earbuds 101A may be changed. In one embodiment, at least one of the earbuds 101A may transmit data to the electronic device. For example, the data may include information for controlling the audio signal output through the earbuds 101A (e.g., information for playing a sound source, information for pausing the sound source, information for stopping the sound source, information for controlling the volume of the sound source, and information for selecting the sound source.

In FIG. 3A, the power supply device 200 may have a structure that can be opened and closed. In one embodiment, in response to the operation of opening the power supply device 200, the power supply device 200 may perform a triggering operation for Bluetooth pairing with the earbuds 101A. In one embodiment, the earbuds 101A is generally small in size and may include a rechargeable battery (e.g., the battery 189) to provide mobility. Accordingly, the earbuds 101A may be stored connected to a separate power supply device 200 to prevent loss while the earbuds 101A is not in use. In one embodiment, the earbuds 101A may be connected to the power supply device 200 and be able to charge the battery while being stored. In one embodiment, the earbuds 101A may include a sensor (e.g., the sensor 176) corresponding to a sensor of the power supply device 200. For example, the power supply device 200 may include a Hall sensor (or hall IC) and the earbuds 101A may include a magnet (for example, included in the sensor 176). In one embodiment, when the earbuds 101A is accommodated in the power supply device 200, the Hall sensor of the power supply device 200 can recognize a magnet in the earbuds 101A.

In one embodiment, the earbuds 101A may include at least one conductive pin pad (e.g., the connection terminal 178) on the outside. In one embodiment, the power supply device 200 may include at least one externally conductive pin (e.g., a conductive terminal). The conductive pin pad of the earbuds 101A and the conductive pin of the power supply 200 may be arranged to physically contact each other while the earbuds 101A is connected to the power supply 200. In one embodiment, when the earbuds 101A is connected to the power supply device 200, the conductive pin of the earbuds 101A and the conductive pin of the power supply device 200 may be in contact and electrically connected. In one embodiment, by identifying a conductive pin contact, the earbuds 101A or the power supply 200 can determine whether the earbuds 101A is connected to the power supply 200. In one embodiment, the earbuds 101A may detect the amount of light reflected from the power supply device 200 through a proximity sensor (e.g., included in the sensor 176).

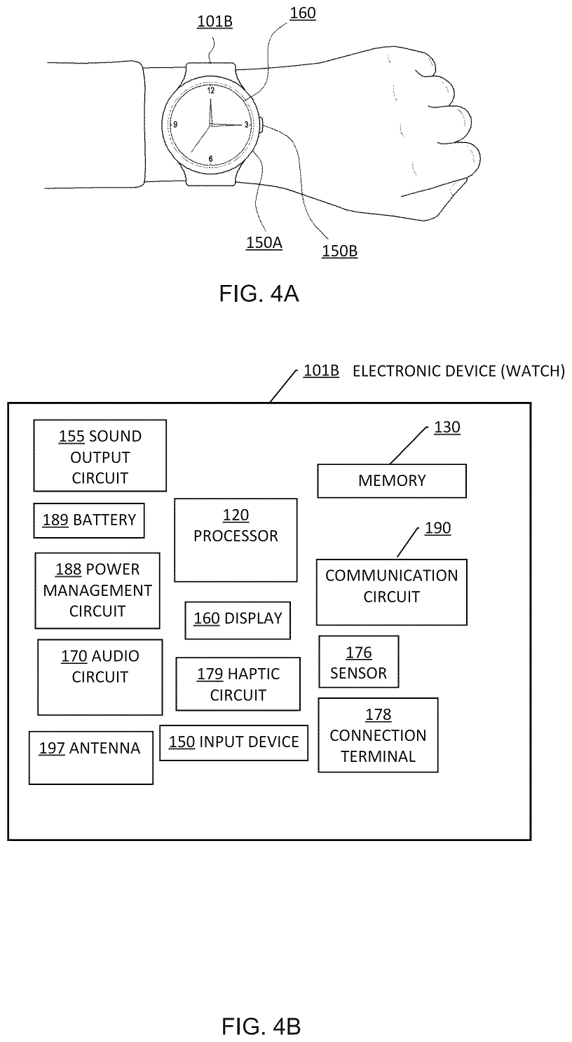

FIG. 4A illustrates a second example of the electronic device 101, which is a watch 101B. FIG. 4B illustrates that the watch 101B may include multiple components, such as the processor 120, the memory 130, the input device 150 (150A and 150B), the sound output circuit 155, the display 160, the audio circuit 170, the sensor 176, the connection terminal 178, the haptic circuit 179, the power management circuit 188, the battery 189, and the communication circuit 190. Those components shown in FIG. 3B are examples, that is, some of those components may be omitted or other components (for example, shown in FIG. 2) may be added to the watch 101B.

In some embodiments, the watch 101B may be paired with a computer, an electronic device, or a mobile device that corresponds to the (first) electronic device 101 shown in FIG. 2. That is, any data processed by the watch 101B can be shown on the display 160 of the (first) electronic device 101. In some embodiments, some operations can be shared between the watch 101B and the (first) electronic device 101.

In FIG. 4A, the watch 101B may include a first input device 150A, a second input device 150B, and the display (touch screen) 160. The watch 101B may be implemented as a smart watch. The processor 120 of the watch 101B may control the overall operation of the watch 101B.

The watch 101B may display data or information through the display 160. The watch 101B may display data stored in the memory 130 through the display 160. For example, the watch 101B may display data or information provided by at least one application stored in the memory 130 through the display 160. Further, the watch 101B may display an execution screen or an execution state of at least one application through the display 160.

In one embodiment, the display 160 may display a user interface for performing a function of the watch 101B. For example, the display 160 may display an execution screen of at least one application stored in the watch 101B. Further, the display 160 may simultaneously display execution screens of a plurality of applications stored in the watch 101B.

In one embodiment, through the display 160, the watch 101B may identify at least one of a touch input, drag input (or touch-drag input), swipe input, and pinch input on the display 160.

The first input device 150A may generate an input signal in response to an input to the watch 101B. For example, the first input device 150A may be implemented in the form of a bezel of a watch. The user may rotate the first input device 150A in a first direction (for example, a clockwise direction) or a second direction (for example, a counterclockwise direction), and the watch 101B may identify the input corresponding to the rotation.

The watch 101B may further include the second input device 150B. For example, the second input device 150B may be implemented in the form of a watch stem. The user may rotate the second input device 150B and press the second input device 150B, and the watch 101B may identify the input corresponding to the rotation or pressing.

The watch 101B may further include the sound output circuit 155 (e.g., a speaker). For example, the watch 101B may provide information in the form of a voice or a sound through the speaker 155.

In some embodiments, the execution screen may be a screen on which a corresponding application is executed and that indicates an execution state. That is, the corresponding application has already been executed or is being executed.

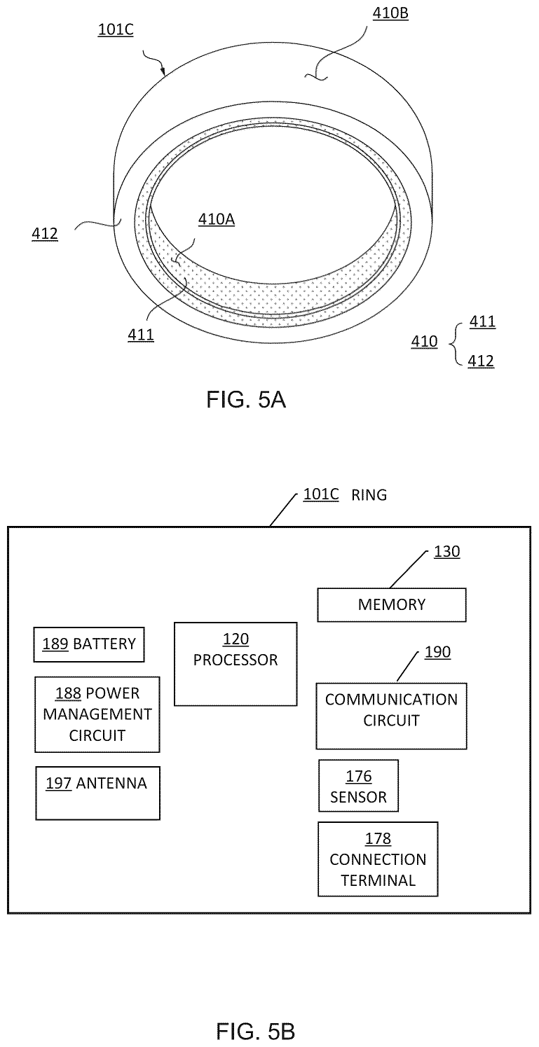

FIG. 5A illustrates a third example of the electronic device 101, which is a ring 101C. FIG. 5B illustrates that the ring 101C may include multiple components.

In FIG. 5A, the ring 101C may include a frame 410. According to an embodiment, the ring 101C may be worn by a user. For example, the ring 101C may be configured to obtain information related to the user through the body part. For example, the ring 101C may provide the user with information indicating a state of the user, based on obtaining the information related to the user. For example, the ring 101C may be configured to display information indicating the state of the user through a display of the ring 101C and/or an external electronic device (e.g., the second electronic device 102 or the third electronic device 104 of FIG. 2) connected to the ring 101C, thereby providing the user with the information indicative of the state of the user. The ring 101C may provide the user with information related to the user wearing the ring 101C via the external electronic device connected to the ring 101C.

In some embodiments, the ring 101C may be paired with a computer, an electronic device, or a mobile device that corresponds to the (first) electronic device 101 shown in FIG. 2. That is, any data processed by the ring 101C can be shown on the display 160 of the (first) electronic device 101. In some embodiments, some operations can be shared between the ring 101C and the (first) electronic device 101.

For example, the body part of the user (on which the ring 101C is worn) may be the user's finger. For example, the frame 410 of the ring 101C may have a ring shape, such that the ring 101C may be worn on the user's finger. However, the disclosure is not limited thereto. The ring 101C, which may be referred to as a wearable device, may have a shape corresponding to the body part of the user.

According to an embodiment, the frame 410 may include a first surface 410A (e.g., an inner surface) facing the body part of the user while the ring 101C is worn on the body part of the user, and a second surface 410B (e.g., an outer surface) opposite to the first surface 410A. The body part may be one of the user's fingers. For example, when the ring 101C is worn by the user, at least a portion of the first surface 410A may contact the body part of the user. For example, the first surface 410A may surround the body part of the user wearing the ring 101C. For example, the first surface 410A may cover the body part of the user wearing the ring 101C. For example, the first surface 410A may be configured such that the ring 101C is fastened to the body part by pressurizing the body part of the user when the ring 101C is worn by the user.

For example, the second surface 410B may be an external appearance of ring 101C together with the first surface 410A. For example, the second surface 410B may be a ring-shaped frame 410 together with the first surface 410A. The second surface 410B (opposite to the first surface 410A) may be farthest from the body part. For example, the first surface 410A may be an inner circumferential surface of the frame 410. The second surface 410B (opposite to the first surface 410A) may be an outer circumference surface of the frame 410.

Here, the ring 101C has been described as being worn on a body part of the user, but the disclosure is not limited thereto. The term “body part” is used to describe the body part of the user on which the ring 101C is worn and is not intended to limit the body part of the user on which the ring 101C is worn or a positional relationship between the body part and the ring 101C thereto. For example, the body part may be one of the user's fingers, but is not limited thereto.

According to an embodiment, the frame 410 may include a first frame 411 defining the first surface 410A, and a second frame 412 defining the second surface 410B and coupled to the first frame 411.

For example, the first frame 411 may be a portion of the frame 410 including the first surface 410A. For example, when the ring 101C is worn by the user, the first frame 411 may be in contact with the body part of the user. The first frame 411 may include at least one of silicon, epoxy, and acryl, but is not limited thereto.

For example, the second frame 412 may surround the first frame 411. For example, the second frame 412 may support the first frame 411. For example, the second frame 412 may form the outer appearance of the frame 410 together with the first frame 411. For example, the second frame 412 may be a portion of the frame 410 including a second surface 410B opposite to the first surface 410A. The second frame 412 may include at least one of metal and titanium, but is not limited thereto. The frame 410 of the ring 101C may include the first frame 411 and the second frame 412 including different materials, thereby providing a user with various user experiences.

In FIG. 5B, the ring 101C may include multiple components in the frame 410 to perform functions of the ring 101C. For example, the ring 101C may include the processor 120, the memory 130, the sensor 176, the battery 189, the communication circuit 190, the power management circuit 188, and the antenna 197. For example, those components of the ring 101C may be included in the first frame 411. The components included in the ring 101C are not limited to the above-described components.

In some embodiments, the sensor 176 may include at least one of a temperature sensor, a proximity sensor, a motion sensor, or a pressure sensor. Further, the sensor 176 may include a light emitter facing the first surface 410A of the frame 410 and a light receiver spaced apart from the light emitter. In some embodiments, the sensor 176 is configured to detect biometric information about the user.

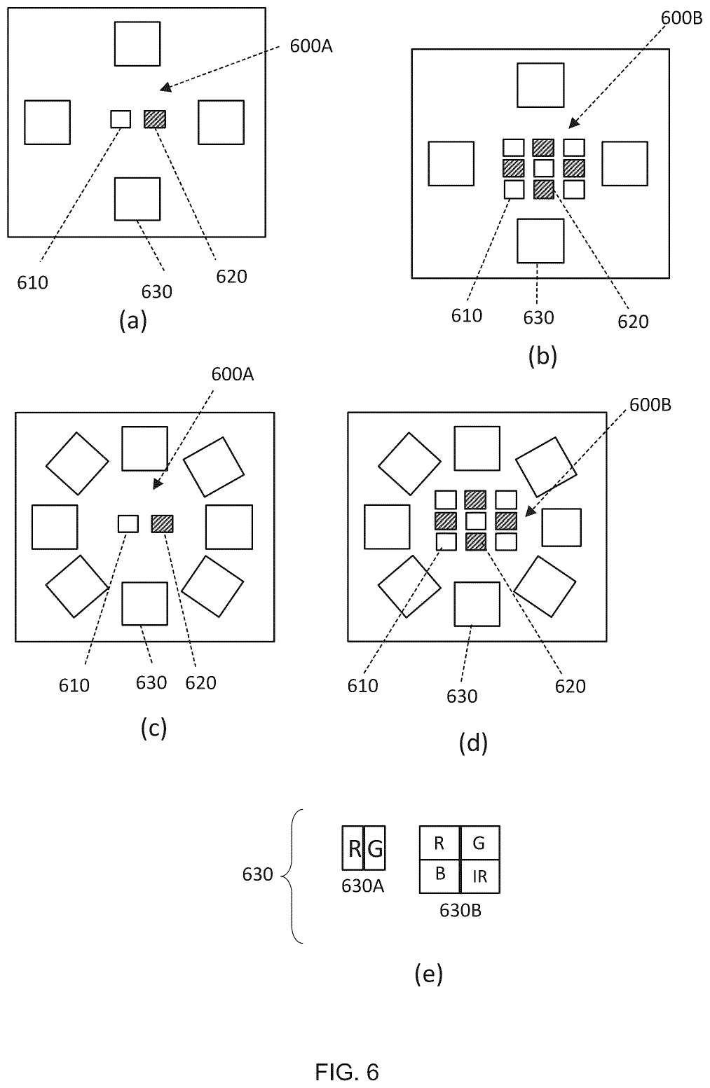

FIG. 6 illustrates example structures of a sensor for detecting biometric information according to one or more example embodiments. The sensor of FIG. 6 may correspond to the sensor 176 of FIGS. 2, 3B, 4B, and 5B.

In (a) of FIG. 6, a light emission portion 600A may include a first light emission element 610 and a second light emission element 620. A plurality of light-receiving devices 630 may be provided around or near the light emission portion 600A. For example, four light-receiving devices 630 may be provided, one on each of the upper, lower, left, and right sides of the light emission portion 600A. In other words, the light-receiving devices 630 may be arranged in at least four positions around the light emission portion 600A. However, embodiments of the present disclosure are not limited thereto; for example, two light-receiving devices 630 may be provided, one on each of the opposite sides of the light emission portion 600A. Photodiodes, phototransistors, or charge-coupled devices (CCDs) may be used as the light-receiving devices 630. The light emission portion 600A and the light-receiving devices 630 may be provided on or in a substrate (e.g., a PCB).

In (a) of FIG. 6, the light emission portion 600A may include a plurality of first light emission elements 610 and a plurality of second light emission elements 620, and (b) of FIG. 6 illustrates an example thereof.

In (b) of FIG. 6, a light emission portion 600B may include a two-dimensional (2D) array in which a plurality of first light emission elements 610 and a plurality of second light emission elements 620 are mixed.

Also, according to another exemplary embodiment, in the structures (a) and (b) of FIG. 6 may be modified to arrange a plurality of light-receiving devices 630 in an annular array around the light emission portions 600A and 600B, and (c) and (d) of FIG. 6 illustrate examples thereof.

Each of the light-receiving devices 630 shown in (a)-(d) of FIG. 6 may be implemented as a single photodiode, or as two or more photodiodes as shown in (e) of FIG. 6. Referring to (e) of FIG. 6, a light-receiving device 630A may include two light-receiving devices configured to receive optical signals having different wavelength ranges, such as an optical signal in a red R region, and an optical signal in a green G region. In this case, one of the first emission element 610 and the second light emission element 620 may be a red light source, and the other one may be a green light source. However, the light emission wavelengths of the first emission element 610 and the second light emission element 620 and the light-receiving device 630A are not limited to these specific wavelengths and may be varied in various ways.

The structure of the light-receiving device 630 may be modified to have the structure of a light-receiving device 630B as illustrated in (e) of FIG. 6. The light-receiving device 630B may include four light-receiving devices configured to receive optical signals of different wavelength ranges. For example, the four light-receiving devices may be configured to receive optical signals in the wavelength ranges of red R, green G, blue B, and infrared IR light, respectively. The light emission wavelengths of the light emission portions 600A and 600B may be configured to correspond with the wavelength ranges of the received optical signals.

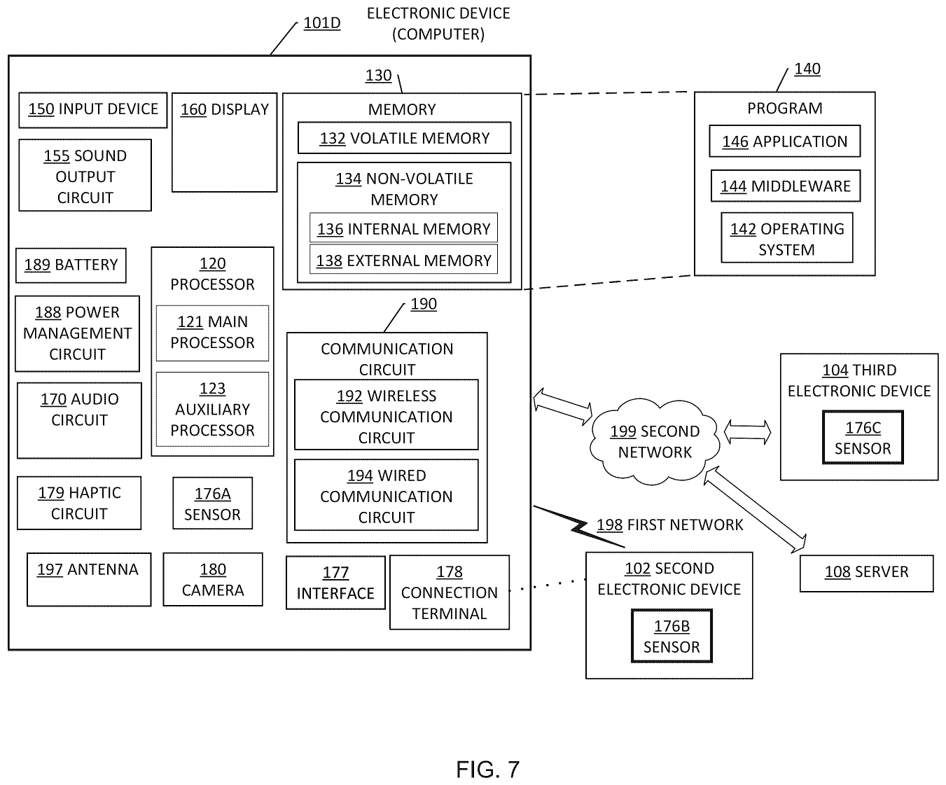

FIG. 7 illustrates an example system having a fourth example (a computer) of the electronic device with remotely located electronic devices and their sensors in accordance with some embodiments of the disclosure. The electronic device (computer) 101D may include multiple components such as the processor 120 and the memory 130, which are described in the above paragraphs. Other components are described above with respect to FIG. 2.

In FIG. 7, the remotely located electronic devices, namely, the second electronic device 102 and the third electronic device 103 may include their respective sensors, the sensor 176B and the sensor 176C, which may respectively correspond to the sensor illustrated in FIG. 6.

In one case where a user is located at or near the second electronic device 102, the sensor 176B of the second electronic device 102 may be used to detect signals (e.g., biometric signals) from the user. Then, the second electronic device 102 may transmit the detected signals to the electronic device (computer) 101D over the first network 198. Next, the transmitted signals may be (temporarily or permanently) stored in the memory 130 and the processor 120 may perform various functions regarding the signals originally generated from the sensor 176B of the second electronic device 102.

Similarly, in another case where a user is located at or near the third electronic device 104, the sensor 176C of the third electronic device 104 may be used to detect signals (e.g., biometric signals) from the user. Then, the third electronic device 104 may transmit the detected signals to the electronic device (computer) 101D over the second network 199. Next, the transmitted signals may be (temporarily or permanently) stored in the memory 130 and the processor 120 may perform various functions regarding the signals originally generated from the sensor 176C of the third electronic device 104.

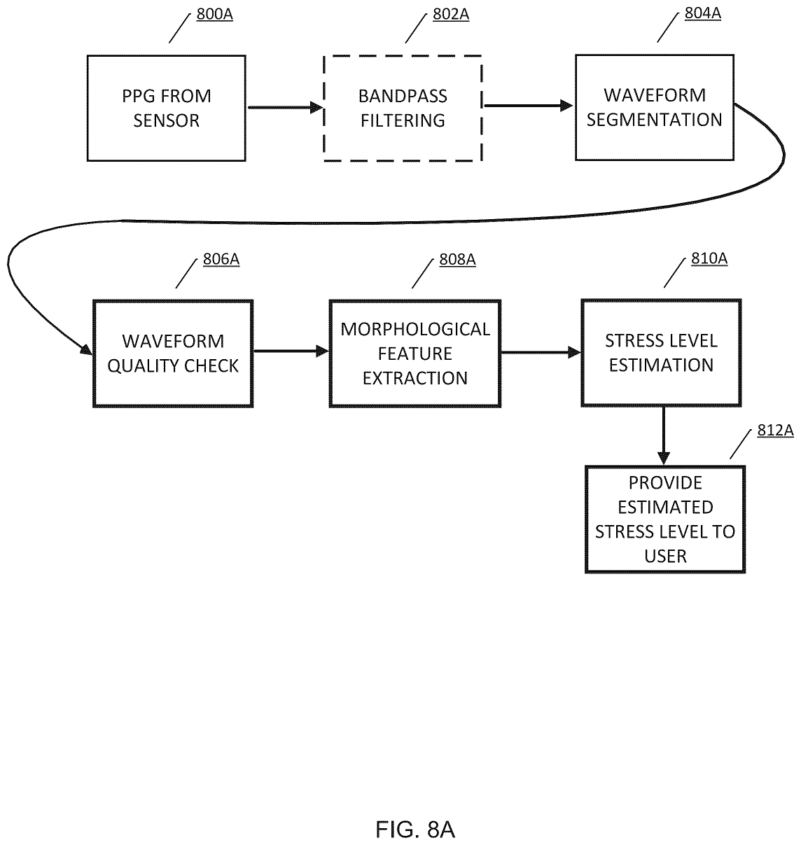

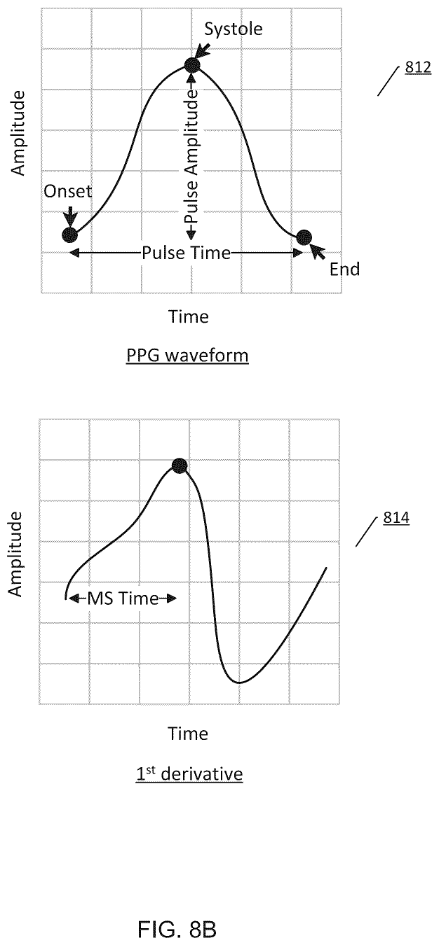

FIG. 8A illustrates a series of operations (a “pipeline”) 800A to 812A performed by electronic devices, for example, the electronic devices described above and shown in FIGS. 2 to 7. For example, the (first) electronic device 101 of FIG. 2 may perform the series of operations of FIG. 8A. In some embodiments, the processor 120, the memory 130, and/or the combination of the processor 120, the memory 130 may perform some or all of operations 800A to 812A. In some embodiments, some of operations 800A to 812A may be software codes stored in any of the memory blocks (132, 134, 136, or 138) of the memory 130. In some embodiments, some of operations 800A to 812A may correspond to dedicated hardware blocks contained in any of the processor blocks (121 or 123) of the processor 120. The disclosure is not limited to the above described embodiments. For example, in some embodiments, operations 800A to 812A may be performed by, implemented in, or stored in any other components of the (first) electronic device 101. In some embodiments, operations 800A to 812A may be performed by, implemented in, or stored in the components of the electronic device (earbuds) 101A, the electronic device (watch) 101B, or the electronic device (ring) 101C.

In some embodiments, operations 800A to 812A may be performed by, implemented in, or stored in the components of the combinations of 1) the (first) electronic device 101 and the electronic device (earbuds) 101A; 2) the (first) electronic device 101 and the electronic device (watch) 101B; or 3) the (first) electronic device 101 and the electronic device (ring) 101C.

In some embodiments, operations 800A to 812A may be performed by, implemented in, or stored in the components of the electronic device (computer) 101D, the second electronic device 102, or the third electronic device 104.

In FIG. 8A, operation 800A is to receive PPG signals about a user from a sensor. FIG. 8B shows a “PPG waveform” 812, which is an example waveform of one of the PPG signals.

Operation 802A is to perform a bandpass filtering of the PPG signals received at operation 802A. Operation 802A is to filter out, using a bandpass filter, noise of the PPG signals received at operation 802A. In an embodiment, the bandpass filter may pass through signals within a particular range, for example, from 0.7 Hz to 3.5 Hz, in order to remove the noise (signal components outside the heartbeat spectrum). Operation 802A may be optional.

Operation 804A is to perform a waveform segmentation. In other words, operation 804A is to segment waveforms of the PPG signals filtered by the bandpass filter at operation 802A. In some embodiments, the PPG signals may be segmented into 1-minute non-overlapping chunks.

Operation 806A is to perform a waveform quality check. In other words, operation 806A is to check qualities of the waveforms segmented at operation 804A. For example, as shown in FIG. 8B, troughs of the PPG waveform 812 are detected and a peak of the first derivative 814 of the PPG waveform 812 is detected. Thus, the troughs of the PPG waveform 812 and the peak of the first derivative 814 of the PPG waveform 812 (marked as black circles in FIG. 8B) are extracted as fiducial points.

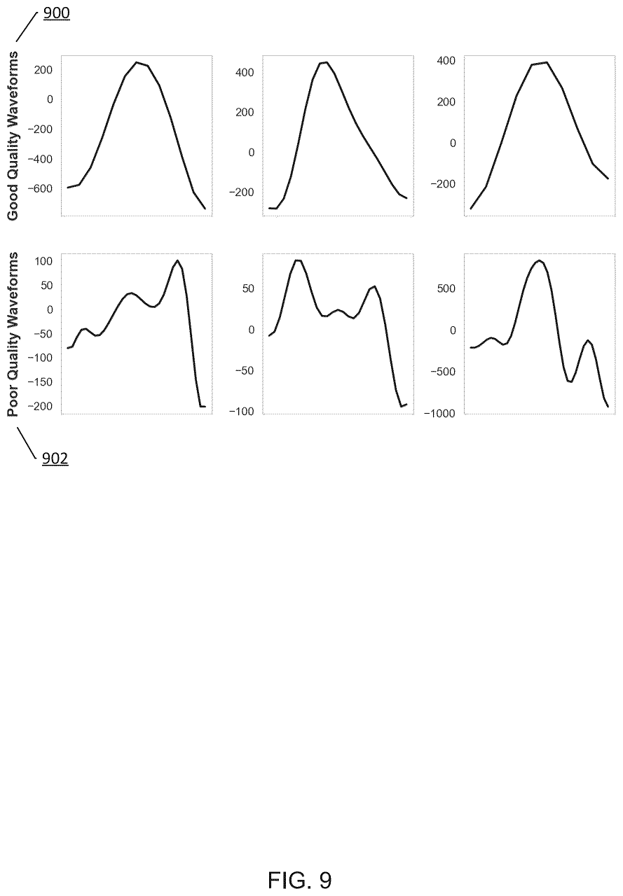

By extracting the fiducial points, a quality criterion may be developed based on a number of maxima detected in the original waveform (the PPG waveform 812). In some embodiments, because only the systolic peak is expected in PPG signals' waveforms, waveforms with multiple detected peaks are treated as poor quality waveforms, and thus, the waveforms with multiple detected peaks are excluded from analysis. FIG. 9 illustrates waveforms 900, which are examples of good quality waveforms passed the waveform quality check at operation 806A. Also, FIG. 9 illustrates waveforms 902 that are examples of poor quality waveforms that do not pass the waveform quality check at operation 806A.

Following this process of operation 806A, features for the remaining waves of acceptable quality may be extracted, which would efficiently and effectively take advantage of the low-fidelity waveforms sampled by resource-constrained electronic devices (such as wearable devices shown in FIGS. 3 to 6.

Operation 808A is to perform morphological feature extraction. In other words, operation 808A is to extract morphological features of the waveforms processed in operation 806A. The morphological features extracted at operation 808A correspond to fiducial points such an onset, an end, and a systolic peak of a waveform (regarding a PPG signal related to a user); a maximum upslope of a first derivative of the waveform. For example, the PPG waveform 812 of FIG. 8B includes the onset (“onset”), the end (“End”), and the systolic peak (“Systole”). Also, the first derivate 814 of the PPG waveform 812 has the maximum upslope, which is marked with a black circle in the “1st derivative” waveform.

The extracted morphological features may be robust and reliable features even in low-fidelity resource-constrained sampling conditions. The extracted morphological features may be used to calculate a plurality of time/amplitude factors to be used in later operations, for example, 1) pulse time (time from the onset to the end), 2) crest time (time from the onset to the systolic peak), 3) a maximum slope (MS) time (time from the onset to the maximum upslope of first derivative), 4) pulse (wave) amplitude (change in amplitude from the onset to the systolic peak), or 5) MS amplitude (change in amplitude from the onset to the maximum upslope of the first derivative). FIG. 8B show some of those time/amplitude factors (‘pulse time’, ‘pulse amplitude’, and ‘MS time’.)

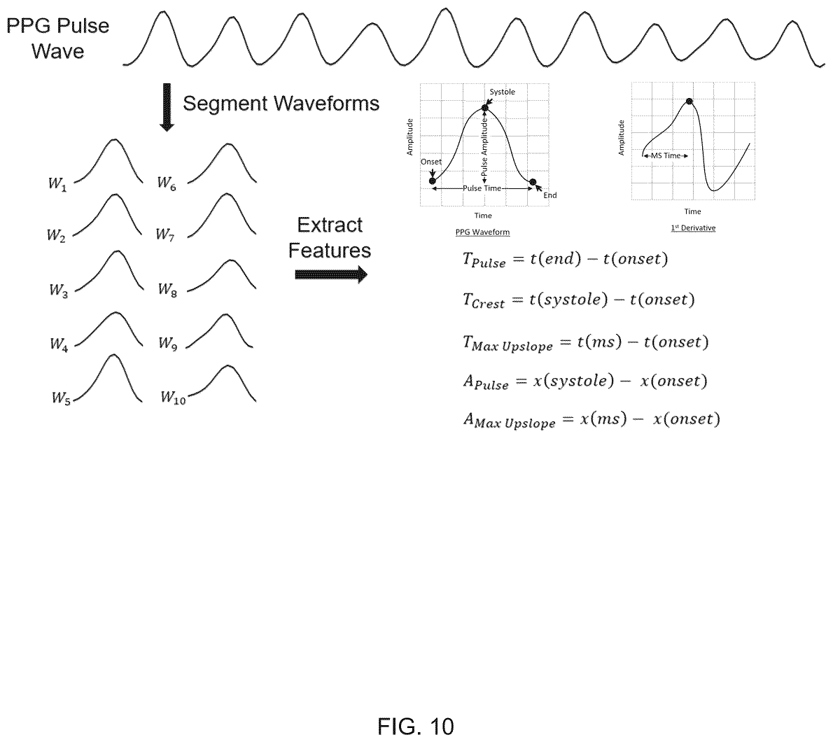

FIG. 10 illustrates an example waveform (“PPG Pulse Wave”) of the PPG signal about a user. For example, by operation 804A, the PPG signal' waveform is segmented into ten different waveforms (W₁ to W₁₀), as shown in FIG. 10. Then, one of the segmented waveforms (for example, W₁) may be analyzed to extract the above described the plurality of time/amplitude factors: 1) pulse time (time from the onset to the end, Tₚᵤₗₛₑ); 2) crest time (time from the onset to the systolic peak, T_{crest}); 3) MS time (time from the onset to the maximum upslope of first derivative): T_{Max Upslope}); 4) pulse (wave) amplitude (change in amplitude from the onset to the systolic peak, Aₚᵤₗₛₑ) or 5) MS amplitude (change in amplitude from the onset to the maximum upslope of the first derivative, A_{Max Upslope}). In some embodiments, other segmented waveforms may be analyzed to extract their respective time/amplitude factors.

Operation 810A is to perform stress level estimation. In other words, operation 810A is to estimate stress level of the user based on the morphological features extracted in operation 808A.

The morphological features may be selected in such a way as to enable the stress level estimation. In some embodiments, the stress level estimation may be performed using an artificial intelligence model such as a random forest model.

In some embodiments, the artificial intelligence model receives the above described time/amplitude factors as training data. For example, there may be two classes of data: non-stress class data (e.g., baseline data and music recovery data) and stress class data (e.g., mental arithmetic data and public speaking data). The respective time/amplitude factors of those two classes of data are input to the artificial intelligence model (e.g., the random forest model) for training and, for example, leave one subject out cross validation is performed. For example, performance of the training of the artificial intelligence model is accessed or evaluated using F1 score as well as balanced accuracy, precision, or recall of the artificial intelligence model.

After the training of the artificial intelligence is completed, the trained artificial intelligence model receives new sets of the time/amplitude factors and produces a stress level estimation. That is, the artificial intelligence model determines whether a user's PPG signals (having the new sets of the time/amplitude factors) represent a stress status of the user.

Operation 812A is to provide the estimated stress level to a user. In some embodiments, the estimated stress level may be numerical values, such as zero to 100 values (e.g., 75) or percentage (e.g., 75%). In some embodiments, the estimated stress level may be provided to the user via an audio signal (e.g., spoken sentence: “Your estimated stress level is 75 percent.”) or through a display.

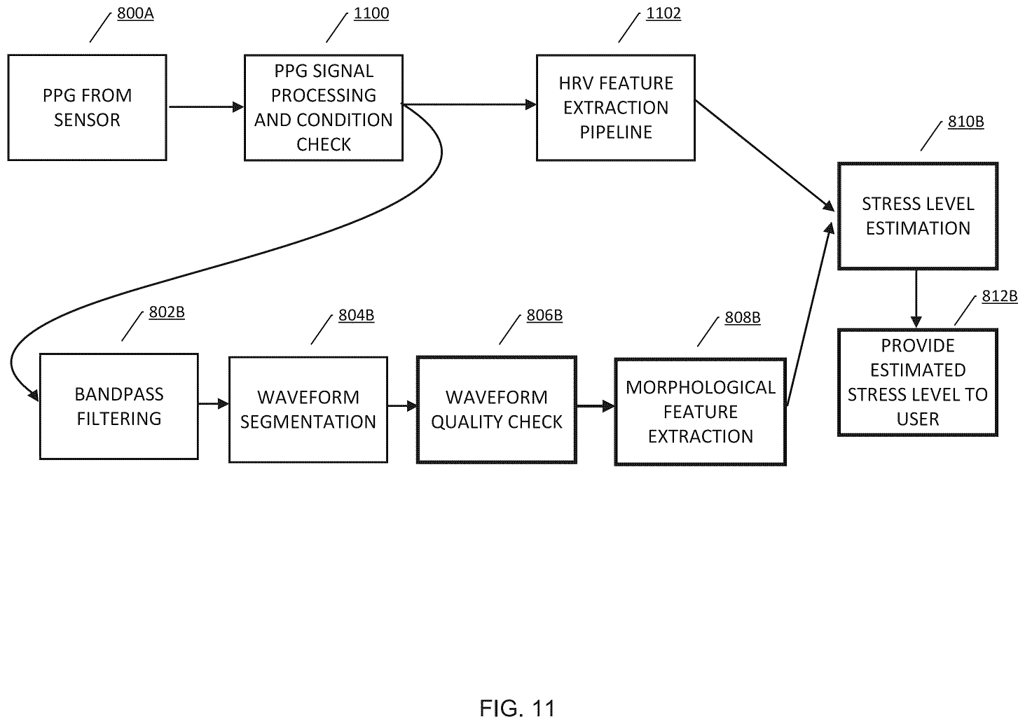

FIG. 11 illustrates a configuration of combining the HRV feature extraction pipeline (e.g., shown in FIG. 1 and described above) with the PPG morphological feature extraction pipeline (e.g., shown in FIG. 8A and described above).

FIG. 11 shows operation 1100 where the PPG signals are processed and their conditions are checked. When a condition is met, the HRV feature extraction pipeline 1102 may be used for stress level estimation. If the condition is not met, the PPG morphological feature extraction pipeline (operations 804B, 806B, 808B) are performed. For example, the condition may be a case whether the user is stationary when the PPG signals are detected, thus the PPG signals have relatively low level of noises. That is, based on the level of noises in the PPG signals, the HRV feature extraction pipeline 1102 (or, alternatively, the PPG morphological feature extraction pipeline) is used for the stress level estimation. Operations 804B, 806B, 808B, and 812B of FIG. 11 may be identical to or correspond to operations 804A, 806A, 808A, and 812B of FIG. 8A.

A first particular application (a mobile phone) is described below.

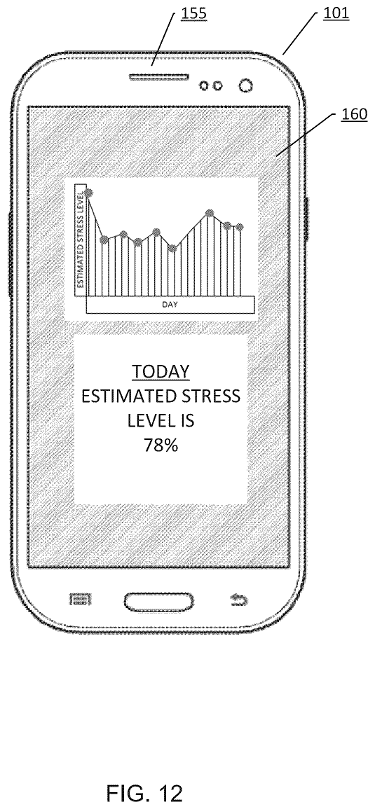

As shown in FIG. 12, the (first) electronic device 101 may be a mobile device (such as a smart phone). In some embodiments, the (first) electronic device 101 may perform operations illustrated in FIG. 8A and described above.

At operation 800A, the (first) electronic device 101 (e.g., the processor 120) may receive a PPG signal about a user from the sensor 176. The received PPG signal may be stored in the memory 130.

At operation 802A, the (first) electronic device 101 may utilize a bandpass filter to filter out some unnecessary bandwidths of the stored PPG signal. For example, the bandpass filter may be included in the processor 120, the memory 130, or the program 140. The filtered out PPG signal may be stored in the memory 130.

At operation 804A, the (first) electronic device 101 may divide the output of operation 802A (the filtered out PPG signal) into a plurality of waveforms that may be stored in the memory 130.

At operation 806A, the (first) electronic device 101 may check qualities of the plurality of waveforms.

At operation 808A, the (first) electronic device 101 may extract morphological features of some waveforms that pass the quality check performed at operation 806A.

At operation 810A, the (first) electronic device 101 may estimate a stress level of the user, based on the extracted morphological features, by using a trained artificial intelligence model.

At operation 812A, the (first) electronic device 101 may provide the estimated stress level to the user. As illustrated in FIG. 12, in some embodiments, the (first) electronic device 101 shows the estimated stress level on the display 160 as a text sentence: “TODAY ESTIMATED STRESS LEVEL IS 78%”. In some embodiments, the (first) electronic device 101 may generate an audio signal informing the estimated stress level and provide the audio signal to the user through the sound output circuit 155. As illustrated in FIG. 12, in some embodiments, the (first) electronic device 101 may provide historical (e.g., daily) changes of the stress level to the user through the display 160.

A second particular application (a mobile device and earbuds) is described below.

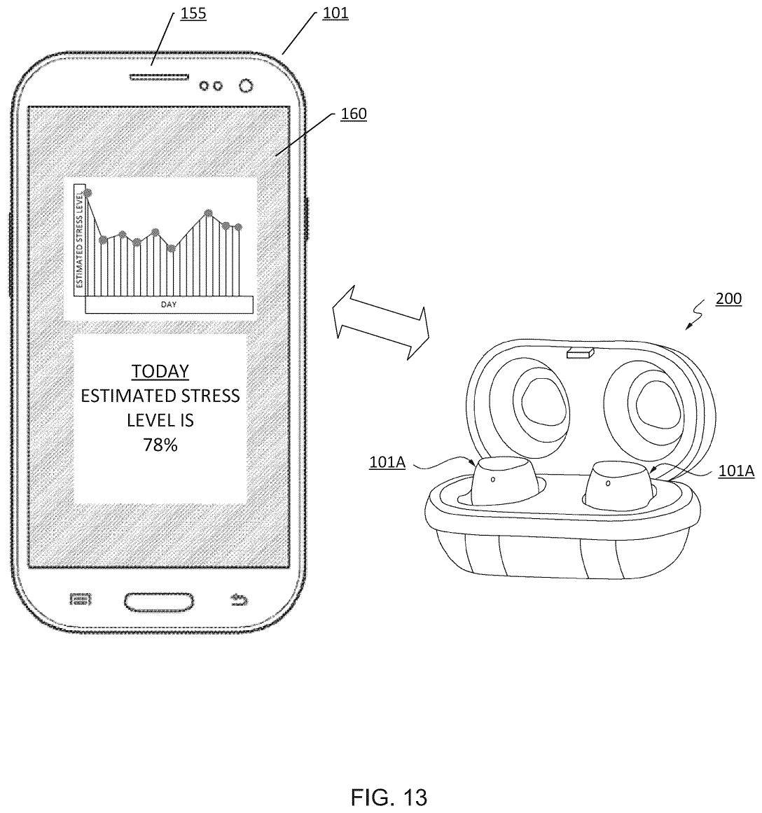

In some embodiments, the electronic device (earbuds) 101A and the (first) electronic device 101 may perform the operations illustrated in FIG. 8A and described above. As illustrated in FIG. 13, in some embodiments, the electronic device (earbuds) 101A may be paired with the (first) electronic device 101 via communication schemes, such as Bluetooth™ or near field communication (NFC).

At operation 800A, the electronic device (earbuds) 101A (e.g., the processor 120 of FIG. 3B) may receive a PPG signal about a user from the sensor 176 of FIG. 3B. The received PPG signal may be stored in the memory 130 of FIG. 3B. In an embodiment, the PPG signal may be transmitted from the electronic device (earbuds) 101A to the (first) electronic device 101, thus the received PPG signal may be stored in the memory 130 of FIG. 2.

At operation 802A, the electronic device (earbuds) 101A may utilize a bandpass filter to filter out some unnecessary bandwidths of the stored PPG signal. For example, the bandpass filter may be included in the processor 120 or the memory 130 of FIG. 3B. The filtered out PPG signal may be stored in the memory 130. In an embodiment, when the PPG signal is stored in the memory 130 of FIG. 2, the (first) electronic device 101 may utilize a bandpass filter to filter out some unnecessary bandwidths of the stored PPG signal. For example, the bandpass filter may be included in the processor 120, the memory 130, or the program 140 of FIG. 2.

At operation 804A, the electronic device (earbuds) 101A may divide the output of operation 802A (the filtered out PPG signal) into a plurality of waveforms, which may be stored in the memory 130 of FIG. 3B. In an embodiment, the (first) electronic device 101 may divide the output of operation 802A (the filtered out PPG signal) into a plurality of waveforms, which may be stored in the memory 130 of FIG. 2.

At operation 806A, the electronic device (earbuds) 101A may check qualities of the plurality of waveforms. In an embodiment, the (first) electronic device 101 may check qualities of the plurality of waveforms.

At operation 808A, the electronic device (earbuds) 101A may extract morphological features of some waveforms that pass the quality check performed at operation 806A. In an embodiment, the (first) electronic device 101 may extract morphological features of some waveforms that pass the quality check performed at operation 806A.

At operation 810A, the electronic device (earbuds) 101A may estimate a stress level of the user, based on the extracted morphological features, by using a trained artificial intelligence model. In an embodiment, the estimated stress level of the user may be output via the sound output circuit 155 of FIG. 3B. In an embodiment, the (first) electronic device 101 may estimate a stress level of the user, based on the extracted morphological features, by using a trained artificial intelligence model.

At operation 812A, the (first) electronic device 101 may provide the estimated stress level to the user. As illustrated in FIG. 13, in some embodiments, the (first) electronic device 101 shows the estimated stress level on the display 160 as a text sentence: “TODAY ESTIMATED STRESS LEVEL IS 78%”. In some embodiments, the (first) electronic device 101 may generate an audio signal informing the estimated stress level and provide the audio signal to the user through the sound output circuit 155. As illustrated in FIG. 13, in some embodiments, the (first) electronic device 101 may provide historical (e.g., daily) changes of the stress level to the user through the display 160.

A third particular application (a mobile phone and a watch) is described below.

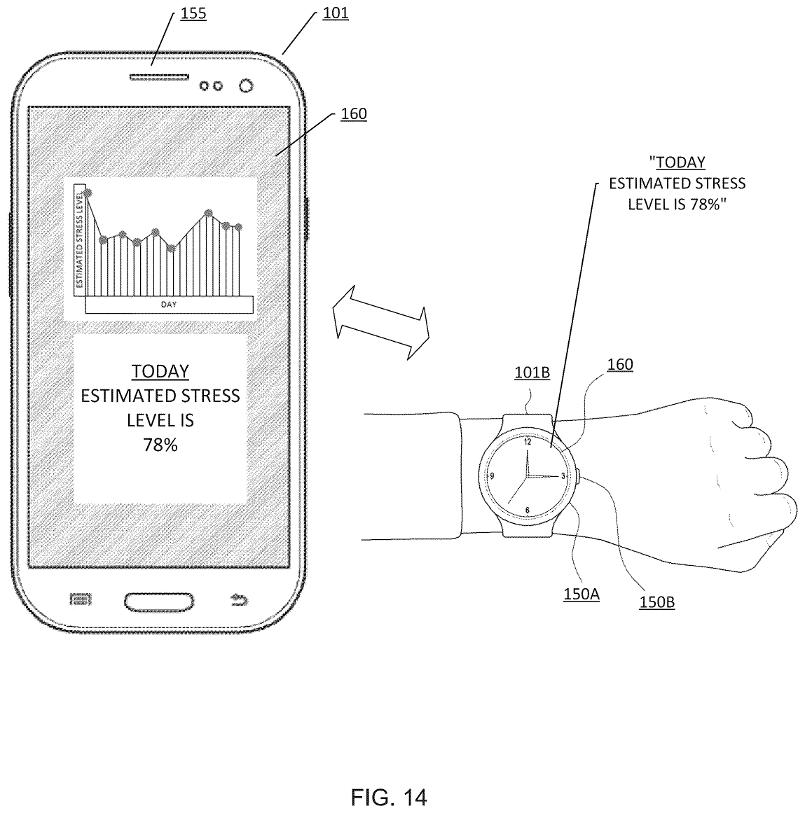

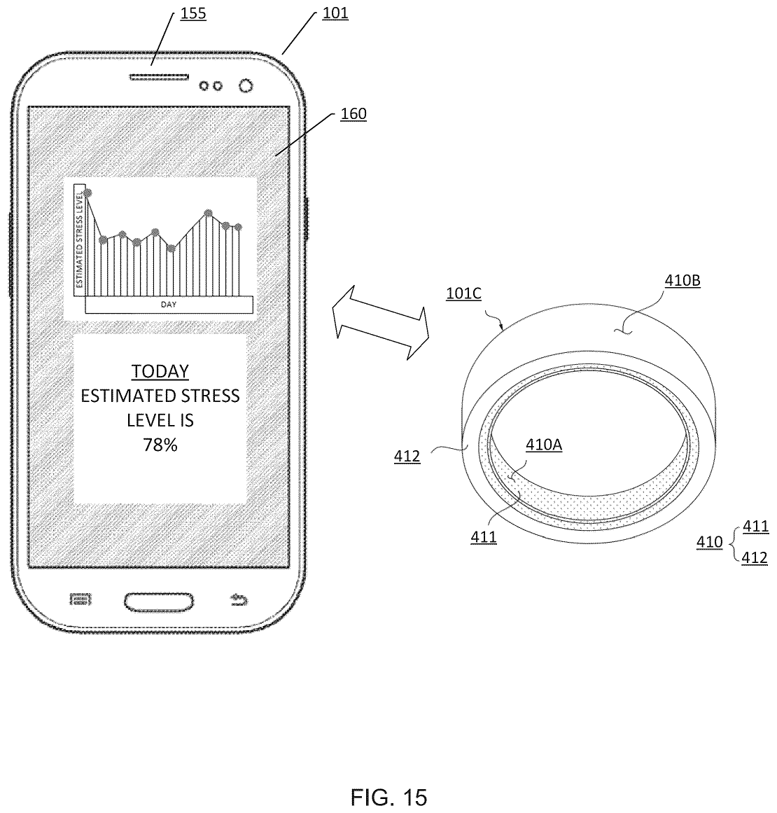

In some embodiments, the electronic device (watch) 101B and the (first) electronic device 101 may perform the operations illustrated in FIG. 8A and described above. As illustrated in FIG. 14, in some embodiments, the electronic device (watch) 101B may be paired with the (first) electronic device 101 via communication schemes, such as Bluetooth™ or NFC.

At operation 800A, the electronic device (watch) 101B (e.g., the processor 120 of FIG. 4B) may receive a PPG signal about a user from the sensor 176 of FIG. 4B. The received PPG signal may be stored in the memory 130 of FIG. 4B. In an embodiment, the PPG signal may be transmitted from the electronic device (watch) 101B to the (first) electronic device 101, thus the received PPG signal may be stored in the memory 130 of FIG. 2.

At operation 802A, the electronic device (watch) 101B may utilize a bandpass filter to filter out some unnecessary bandwidths of the stored PPG signal. For example, the bandpass filter may be included in the processor 120 or the memory 130 of FIG. 4B. The filtered out PPG signal may be stored in the memory 130 of FIG. 4B. In an embodiment, when the PPG signal is stored in the memory 130 of FIG. 2, the (first) electronic device 101 may utilize a bandpass filter to filter out some unnecessary bandwidths of the stored PPG signal. For example, the bandpass filter may be included in the processor 120, the memory 130, or the program 140 of FIG. 2.

At operation 804A, the electronic device (watch) 101B may divide the output of operation 802A (the filtered out PPG signal) into a plurality of waveforms, which may be stored in the memory 130 of FIG. 4B. In an embodiment, the (first) electronic device 101 may divide the output of operation 802A (the filtered out PPG signal) into a plurality of waveforms, which may be stored in the memory 130 of FIG. 2.

At operation 806A, the electronic device (watch) 101B may check qualities of the plurality of waveforms. In an embodiment, the (first) electronic device 101 may check qualities of the plurality of waveforms.

At operation 808A, the electronic device (watch) 101B may extract morphological features of some waveforms that pass the quality check performed at operation 806A. In an embodiment, the (first) electronic device 101 may extract morphological features of some waveforms that pass the quality check performed at operation 806A.

At operation 810A, the electronic device (watch) 101B may estimate a stress level of the user, based on the extracted morphological features, by using a trained artificial intelligence model. In an embodiment, the estimated stress level of the user may be output via the sound output circuit 155 of FIG. 3B. In an embodiment, the (first) electronic device 101 may estimate a stress level of the user, based on the extracted morphological features, by using a trained artificial intelligence model.

At operation 812A, the (first) electronic device 101 may provide the estimated stress level to the user. As illustrated in FIG. 14, in some embodiments, the (first) electronic device 101 shows the estimated stress level on the display 160 (of the (first) electronic device 101) as a text sentence: “TODAY ESTIMATED STRESS LEVEL IS 78%”. In some embodiments, the (first) electronic device 101 may generate an audio signal informing the estimated stress level and provide the audio signal to the user through the sound output circuit 155. As illustrated in FIG. 14, in some embodiments, the (first) electronic device 101 may provide historical (e.g., daily) changes of the stress level to the user through the display 160. In some embodiments, the electronic device (watch) 101B shows the estimated stress level on the display 160 (of the electronic device (watch) 101B) as a text sentence: “TODAY ESTIMATED STRESS LEVEL IS 78%”. In some embodiments, the electronic device (watch) 101B may provide historical (e.g., daily) changes of the stress level to the user through the display 160 of the electronic device (watch) 101B.

A fourth particular application (a mobile phone and a ring) is described below.

In some embodiments, the electronic device (ring) 101C and the (first) electronic device 101 may perform the operations illustrated in FIG. 8A and described above. As illustrated in FIG. 15, in some embodiments, the electronic device (ring) 101C may be paired with the (first) electronic device 101 via communication schemes, such as Bluetooth™ or NFC.

At operation 800A, the electronic device (ring) 101C (e.g., the processor 120 of FIG. 5B) may receive a PPG signal about a user from the sensor 176 of FIG. 5B. The received PPG signal may be stored in the memory 130 of FIG. 5B. In an embodiment, the PPG signal may be transmitted from the electronic device (ring) 101C to the (first) electronic device 101, thus the received PPG signal may be stored in the memory 130 of FIG. 2.

At operation 802A, the electronic device (ring) 101C may utilize a bandpass filter to filter out some unnecessary bandwidths of the stored PPG signal. For example, the bandpass filter may be included in the processor 120 or the memory 130 of FIG. 5B. The filtered out PPG signal may be stored in the memory 130 of FIG. 5B. In an embodiment, when the PPG signal is stored in the memory 130 of FIG. 2, the (first) electronic device 101 may utilize a bandpass filter to filter out some unnecessary bandwidths of the stored PPG signal. For example, the bandpass filter may be included in the processor 120, the memory 130, or the program 140 of FIG. 2.

At operation 804A, the electronic device (ring) 101C may divide the output of operation 802A (the filtered out PPG signal) into a plurality of waveforms, which may be stored in the memory 130 of FIG. 5B. In an embodiment, the (first) electronic device 101 may divide the output of operation 802A (the filtered out PPG signal) into a plurality of waveforms, which may be stored in the memory 130 of FIG. 2.

At operation 806A, the electronic device (ring) 101C may check qualities of the plurality of waveforms. In an embodiment, the (first) electronic device 101 may check qualities of the plurality of waveforms.

At operation 808A, the electronic device (ring) 101C may extract morphological features of some waveforms that pass the quality check performed at operation 806A. In an embodiment, the (first) electronic device 101 may extract morphological features of some waveforms that pass the quality check performed at operation 806A.

At operation 810A, the electronic device (ring) 101C may estimate a stress level of the user, based on the extracted morphological features, by using a trained artificial intelligence model. In an embodiment, the (first) electronic device 101 may estimate a stress level of the user, based on the extracted morphological features, by using a trained artificial intelligence model.

At operation 812A, the (first) electronic device 101 may provide the estimated stress level to the user. As illustrated in FIG. 15, in some embodiments, the (first) electronic device 101 shows the estimated stress level on the display 160 as a text sentence: “TODAY ESTIMATED STRESS LEVEL IS 78%”. In some embodiments, the (first) electronic device 101 may generate an audio signal informing the estimated stress level and provide the audio signal to the user through the sound output circuit 155. As illustrated in FIG. 15, in some embodiments, the (first) electronic device 101 may provide historical (e.g., daily) changes of the stress level to the user through the display 160.

A fifth particular application (a local computer+a remote electronic device) is described below.

In some embodiments, the first electronic device 101 and the second electronic device 102 may perform the operations illustrated in FIG. 8A and described above. As illustrated in FIG. 7, in some embodiments, the first electronic device 101 and the second electronic device 102 via communication schemes, such as WiFi or 3G/4G/5G/6G wireless communication.

At operation 800A, the second electronic device 102 may receive a PPG signal about a user from the sensor 176B of FIG. 7. The received PPG signal may be stored in a memory of the second electronic device 102. In an embodiment, the PPG signal may be transmitted from the second electronic device 102 to the first electronic device 101, thus the received PPG signal may be stored in the memory 130 of the first electronic device 101.

At operation 802A, the second electronic device 102 may utilize a bandpass filter to filter out some unnecessary bandwidths of the stored PPG signal. For example, the bandpass filter may be included in a processor or the memory of the second electronic device 102. The filtered out PPG signal may be stored in the memory of the second electronic device 102. In an embodiment, when the PPG signal is stored in the memory 130 of the first electronic device 101, the first electronic device 101 may utilize a bandpass filter to filter out some unnecessary bandwidths of the stored PPG signal. For example, the bandpass filter may be included in the processor 120, the memory 130, or the program 140 of the first electronic device 101.

At operation 804A, the second electronic device 102 may divide the output of operation 802A (the filtered out PPG signal) into a plurality of waveforms, which may be stored in the memory of the second electronic device 102. In an embodiment, the first electronic device 101 may divide the output of operation 802A (the filtered out PPG signal) into a plurality of waveforms, which may be stored in the memory 130 of the first electronic device 101.

At operation 806A, the second electronic device 102 may check qualities of the plurality of waveforms. In one embodiment, the second electronic device 102 may transmit the plurality of waveforms to the first electronic device 101 after their qualities are checked. In an embodiment, the first electronic device 101 may check qualities of the plurality of waveforms.

At operation 808A, the second electronic device 102 may extract morphological features of some waveforms that pass the quality check performed at operation 806A. In one embodiment, the second electronic device 102 may transmit the extracted morphological features to the first electronic device 101. In an embodiment, the first electronic device 101 may extract morphological features of some waveforms that pass the quality check performed at operation 806A.

At operation 810A, the second electronic device 102 may estimate a stress level of the user, based on the extracted morphological features, by using a trained artificial intelligence model. In one embodiment, the second electronic device 102 may transmit the estimated stress level to the first electronic device 101. In an embodiment, the first electronic device 101 may estimate a stress level of the user, based on the extracted morphological features, by using a trained artificial intelligence model.

At operation 812A, the first electronic device 101 may provide the estimated stress level to the user. As illustrated in FIG. 12, in some embodiments, the first electronic device 101 shows the estimated stress level on the display 160 (of the first electronic device 101) as a text sentence: “TODAY ESTIMATED STRESS LEVEL IS 78%”. In some embodiments, the first electronic device 101 may generate an audio signal informing the estimated stress level and provide the audio signal to the user through the sound output circuit 155. As illustrated in FIG. 12, in some embodiments, the first electronic device 101 may provide historical (e.g., daily) changes of the stress level to the user through the display 160.

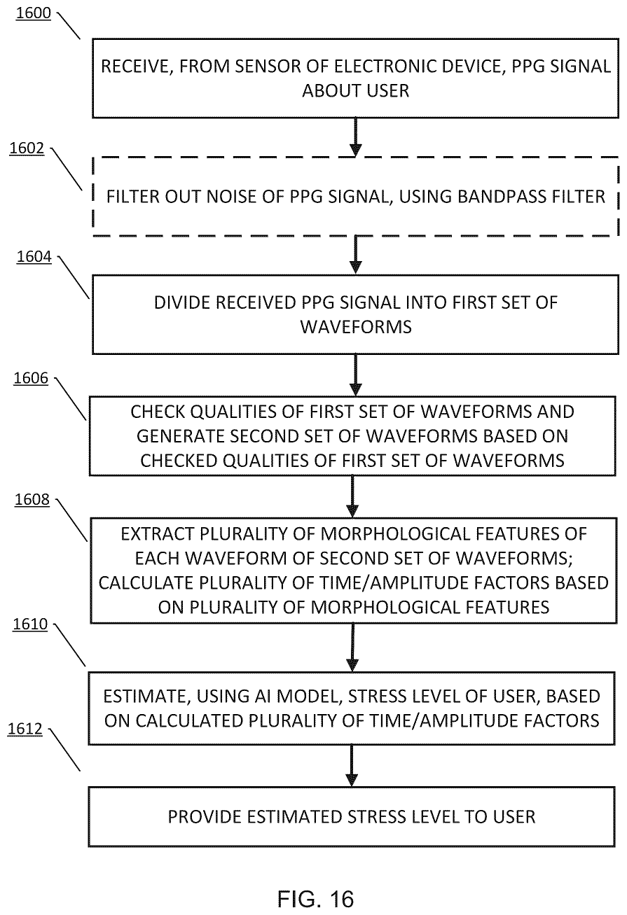

FIG. 16 illustrates a first set of operations in accordance with some embodiments of the disclosure.

Operation 1600 is to receive, from a sensor of an electronic device, a PPG signal about a user. Operation 1600 may correspond to operation 800A of FIG. 8A.

Operation 1602 is to filter out, by using a bandpass filter, noise of the received PPG signal. Operation 1602 may be optional. Operation 1600 may correspond to operation 802A of FIG. 8A.

Operation 1604 is to divide the received PPG signal (or the PPG signal excluding noise by operation 1602) into a first set of waveforms. Operation 1604 may correspond to operation 804A of FIG. 8A.

Operation 1606 is to check qualities of the first set of waveforms and generate a second set of waveforms based on the checked qualities of the first set of waveforms. That is, the second set of waveforms may not include waveforms having poor qualities, among the first set of waveforms. FIG. 9 illustrates examples of good quality waveforms 900 and poor quality waveforms 902. The second set of waveforms may include the good quality waveforms 900. Operation 1606 may correspond to operation 806A of FIG. 8A.

Operation 1608 is to extract a plurality of morphological features of each waveform of the second set of waveforms. For example, the plurality of morphological features may include an onset of the waveform, an end of the waveform, a systolic peak of the waveform, and a maximum upslope of a first derivative of the waveform. For example, the extracted morphological features may be used to calculate a plurality of time/amplitude factors that includes 1) a pulse time instance (time from the onset to the end), 2) a crest time instance (time from the onset to the systolic peak), 3) a MS time instance (time from the onset to the maximum upslope of a first derivative of the waveform), 4) a pulse (wave) amplitude (a change in amplitude from the onset to the systolic peak), or 5) a MS amplitude (a change in amplitude from the onset to the maximum upslope of the first derivative of the waveform). Operation 1608 may correspond to operation 808A of FIG. 8A.

Operation 1610 is to estimate, using an artificial intelligence model, a stress level of the user, based on the calculated plurality of time/amplitude factors. An example of the artificial intelligence model is a random forest model trained with a plurality of training datasets. For example, the plurality of training datasets include a first set of time/amplitude factors about stress tasks and a second set of time/amplitude factors about non-stress tasks. For example, the first set of time/amplitude factors about stress tasks includes mental arithmetic data or public speaking data. For example, the second set of time/amplitude factors about non-stress tasks includes baseline data or music recovery data. Operation 1610 may correspond to operation 810A of FIG. 8A.

Operation 1612 is to provide the estimated stress level to the user. In some embodiments, the estimated stress level may be numerical values, such as zero to 100 values (e.g., 75) or percentage (e.g., 75%). In some embodiments, the estimated stress level may be included in an audio signal that is transmitted to the user. In some embodiments, the estimated stress level may be shown in a display of an electronic device. Operation 1612 may correspond to operation 812A of FIG. 8A.

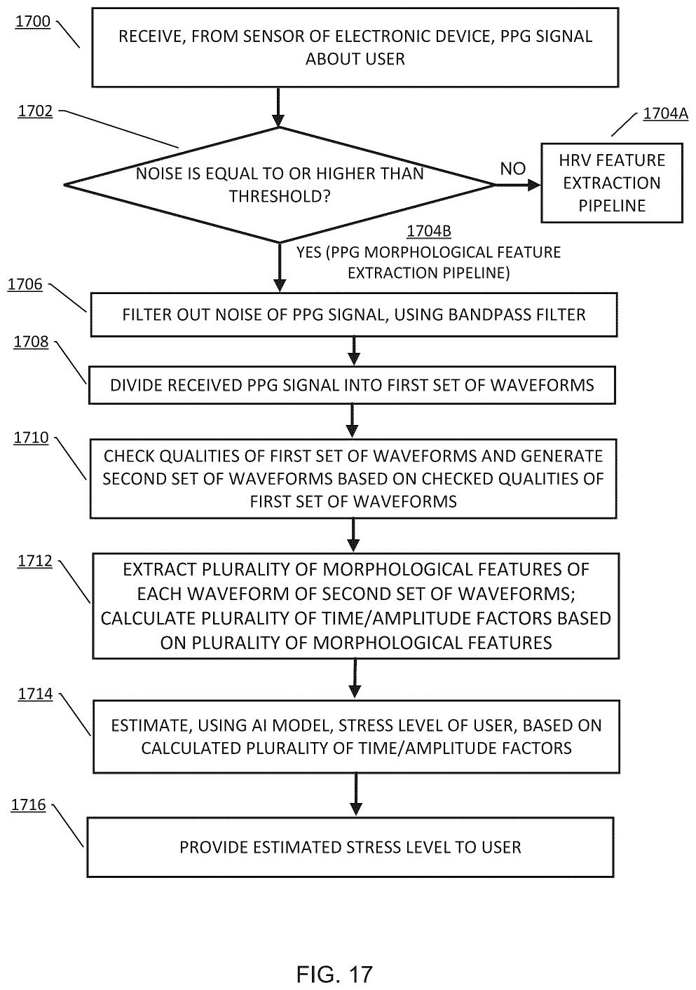

FIG. 17 illustrates a second set of operations in accordance with some embodiments of the disclosure.

Operation 1700 is to receive, from a sensor of an electronic device, a PPG signal about a user. Operation 1700 may correspond to operation 800A of FIG. 8A.

Operation 1702 is to determine whether noise of the received PPG signal is equal to or higher than a threshold.

At operation 1704A, based on a determination that the noise of the received PPG signal is lower than the threshold, the HRV feature extraction pipeline (for example, as shown in FIG. 1 and described above) may be used to extract a plurality of time/amplitude factors of the received PPG signal.

At operation 1704B, Based on the noise of the received PPG signal is equal to or higher than the threshold, the PPG morphological feature extraction pipeline may be used to extract a plurality of time/amplitude factors of the received PPG signal.

Operation 1706 is to filter out, using a bandpass filter, the noise of the received PPG signal. Operation 1706 may correspond to operation 802B of FIG. 11.

Operation 1708 is to divide (segment) the received PPG signal (excluding noise by operation 1706) into a first set of waveforms. Operation 1708 may correspond to operation 804B of FIG. 11.

Operation 1710 is to check qualities of the first set of waveforms and generate a second set of waveforms based on the checked qualities of the first set of waveforms. That is, the second set of waveforms may not include waveforms having poor qualities, among the first set of waveforms. FIG. 9 illustrates examples of good quality waveforms 900 and poor quality waveforms 902. The second set of waveforms may include the good quality waveforms 900. Operation 1710 may correspond to operation 806B of FIG. 11.

Operation 1712 is to extract a plurality of morphological features of each waveform of the second set of waveforms. For example, the plurality of morphological features may include an onset of the waveform, an end of the waveform, a systolic peak of the waveform, and a maximum upslope of a first derivative of the waveform. For example, the extracted morphological features may be used to calculate a plurality of time/amplitude factors that includes 1) a pulse time instance (time from the onset to the end), 2) a crest time instance (time from the onset to the systolic peak), 3) a MS time instance (time from the onset to the maximum upslope of a first derivative of the waveform), 4) a pulse (wave) amplitude (a change in amplitude from the onset to the systolic peak), or 5) a MS amplitude (a change in amplitude from the onset to the maximum upslope of the first derivative of the waveform). Operation 1712 may correspond to operation 80BA of FIG. 11.

Operation 1714 is to estimate, using an artificial intelligence model, a stress level of the user based on the calculated plurality of time/amplitude factors. An example of the artificial intelligence model is a random forest model trained with a plurality of training datasets. For example, the plurality of training datasets include a first set of time/amplitude factors about stress tasks and a second set of time/amplitude factors about non-stress tasks. For example, the first set of time/amplitude factors about stress tasks includes mental arithmetic data or public speaking data. For example, the second set of time/amplitude factors about non-stress tasks includes baseline data or music recovery data. Operation 1714 may correspond to operation 810B of FIG. 11.

Operation 1716 is to provide the estimated stress level to the user. In some embodiments, the estimated stress level may be numerical values, such as zero to 100 values (e.g., 75) or percentage (e.g., 75%). In some embodiments, the estimated stress level may be included in an audio signal that is transmitted to the user. In some embodiments, the estimated stress level may be shown in a display of an electronic device. Operation 1716 may correspond to operation 812A of FIG. 8A.

According to one or more embodiments of the disclosure, a method may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in a machine-readable storage medium, such as memory of the manufacturer's server, a server of the application store, or a relay server.

According to one or more embodiments, each component (e.g., a module or a program) of the above-described components may include a single entity or multiple entities. According to one or more embodiments, one or more of the above-described components may be omitted, or one or more other components may be added. Alternatively or additionally, a plurality of components (e.g., modules or programs) may be integrated into a single component. In such a case, according to one or more embodiments, the integrated component may still perform one or more functions of each of the plurality of components in the same or similar manner as they are performed by a corresponding one of the plurality of components before the integration. According to one or more embodiments, operations performed by the module, the program, or another component may be carried out sequentially, in parallel, repeatedly, or heuristically, or one or more of the operations may be executed in a different order or omitted, or one or more other operations may be added.

According to one or more embodiments, in a non-volatile storage medium storing instructions, the instructions may be configured to, when executed by at least one processor, cause the at least one processor to perform at least one operation. The at least one operation may include displaying an application screen of a running application (e.g., the application 146) on a display (e.g., the display 160), identifying a data input field included in the application screen, identifying a data type corresponding to the data input field, displaying at least one external electronic device, around the electronic device, capable of providing data corresponding to the identified data type, receiving data corresponding to the identified data type from an external electronic device selected from among the at least one external electronic device through a communication circuit, and entering the received data into the data input field.

The embodiments of the disclosure described in the specification and the drawings are only presented as specific examples to easily explain the technical content according to the embodiments of the disclosure and help understanding of the embodiments of the disclosure, not intended to limit the scope of the embodiments of the disclosure. Therefore, the scope of one or more embodiments of the disclosure should be construed as encompassing all changes or modifications derived from the technical spirit of one or more embodiments of the disclosure in addition to the embodiments disclosed herein.

What is claimed is:

1. A computer-implemented method comprising:
receiving, from a sensor of an electronic device, a photoplethysmography (PPG) signal about a user;
dividing the received PPG signal into a first set of waveforms;
checking qualities of the first set of waveforms and generating a second set of waveforms based on the checked qualities of the first set of waveforms;
extracting a plurality of morphological features of each waveform of the second set of waveforms and calculating a plurality of time/amplitude factors based on the extracted plurality of morphological features;
estimating, using an artificial intelligence model, a stress level of the user, based on the calculated plurality of time/amplitude factors; and
providing the estimated stress level to the user.

2. The computer-implemented method of claim 1, wherein the electronic device corresponds to at least one of a mobile phone, earbuds, a watch, a ring, or a remotely located electronic device operatively connected with another electronic device over a communication channel.

3. The computer-implemented method of claim 1, further comprising filtering out, by using a bandpass filter, noise of the received signal before the received PPG signal is divided into the first set of waveforms.

4. The computer-implemented method of claim 1, wherein the checking qualities of the first set of waveforms comprises determining a subset of waveforms not having multiple peaks, and
wherein the generating the second set of waveforms comprises generating the second set of waveforms comprising the subset of waveforms.

5. The computer-implemented method of claim 1, wherein the plurality of morphological features comprise at least two of an onset of a waveform, an end of the waveform, a systolic peak of the waveform, or a maximum upslope of a first derivative of the waveform.

6. The computer-implemented method of claim 5, wherein the plurality of time/amplitude factors comprise at least one of:
a pulse time instance that corresponds to time from the onset to the end,
a crest time instance that corresponds to time from the onset to the systolic peak,
a maximum slope (MS) time instance that corresponds to time from the onset to the maximum upslope of the first derivative of the waveform,
a pulse wave amplitude that corresponds to a change in amplitude from the onset to the systolic peak, or
a MS amplitude that corresponds to a change in amplitude from the onset to the maximum upslope of the first derivative of the waveform.

7. The computer-implemented method of claim 1, wherein the artificial intelligence model is a random forest model trained with a plurality of training datasets.

8. An electronic device comprising:
a sensor;
at least one memory;
at least one processor operatively connected with the sensor and the at least one memory, the at least on processor configured to:
receiving, from the sensor, a photoplethysmography (PPG) signal about a user;
divide the received PPG signal into a first set of waveforms;
check qualities of the first set of waveforms and generate a second set of waveforms based on the checked qualities of the first set of waveforms;
extract a plurality of morphological features of each waveform of the second set of waveforms and calculate a plurality of time/amplitude factors based on the extracted plurality of morphological features;
estimate, using an artificial intelligence model, a stress level of the user, based on the calculated plurality of time/amplitude factors; and
provide the estimated stress level to the user.

9. The electronic device of claim 8, wherein the electronic device corresponds to at least one of a mobile phone, earbuds, a watch, a ring, or a remotely located electronic device operatively connected with another electronic device over a communication channel.

10. The electronic device of claim 8, further comprising a bandpass filter configured to filter out noise of the received signal before the received PPG signal is divided into the first set of waveforms.

11. The electronic device of claim 8, wherein the at least one processor is further configured to:
determine a subset of waveforms not having multiple peaks, and
generate the second set of waveforms comprising the subset of waveforms.

12. The electronic device of claim 8, wherein the plurality of morphological features comprise at least two of an onset of a waveform, an end of the waveform, a systolic peak of the waveform, or a maximum upslope of a first derivative of the waveform.

13. The electronic device of claim 8, wherein the plurality of time/amplitude factors comprise at least one of:
a pulse time instance that corresponds to time from an onset of the waveform to an end of the waveform,
a crest time instance that corresponds to time from the onset of the waveform to an systolic peak of the waveform,
a maximum slope (MS) time instance that corresponds to time from the onset of the waveform to a maximum upslope of a first derivative of the waveform,
a pulse wave amplitude that corresponds to a change in amplitude from the onset of the waveform to the systolic peak of the waveform, or
a MS amplitude that corresponds to a change in amplitude from the onset of the waveform to the maximum upslope of the first derivative of the waveform.

14. The electronic device of claim 8, wherein the artificial intelligence model is a random forest model trained with a plurality of training datasets.

15. A computer-implemented method comprising:
receiving, from a sensor of an electronic device, a photoplethysmography (PPG) signal about a user;
determining whether a noise of the PPG signal is equal to or higher than a threshold;
based on a determination that the noise of the PPG signal is equal to or higher than the threshold, dividing the received PPG signal into a first set of waveforms;
checking qualities of the first set of waveforms and generating a second set of waveforms based on the checked qualities of the first set of waveforms;
extracting a plurality of morphological features of each waveform of the second set of waveforms and calculating a plurality of time/amplitude factors based on the extracted plurality of morphological features;
estimating, using an artificial intelligence model, a stress level of the user, based on the calculated plurality of time/amplitude factors; and
providing the estimated stress level to the user.

16. The computer-implemented method of claim 15, wherein the plurality of morphological features comprise at least two of an onset of a waveform, an end of the waveform, a systolic peak of the waveform, or a maximum upslope of a first derivative of the waveform.
